(19) United States
(12) Patent Application Publication
(10) Pub. No.: US 2007/0009970 A1
(43) Pub. Date: Jan. 11, 2007

(54) Biological patterns for diagnosis and treatment of cancer

(75) Inventors: Jonathan C. Heller, San Francisco, CA (US); Michael Brown, San Francisco, CA (US); David Perry de Valpine, San Francisco, CA (US); Hans Bitter, San Francisco, CA (US); Kathy Stults, Redwood City, CA (US); Isabelle Guyon, Berkeley (FR); Robert Tibshirani, Palo Alto, CA (US)

Correspondence Address: WILSON SONSINI GOODRICH & ROSATI, 650 PAGE MILL ROAD, PALO ALTO, CA, 94304-1050, US

(73) Assignee: Predicant Biosciences, Inc., South San Francisco, CA (US)

(21) Appl. No.: 11/178,245

(22) Filed: Jul. 8, 2005

(51) Int. Cl. G01N 33/574; G06F 19/00

(52) U.S. Cl. 435/7.23; 436/86; 702/19

(57) ABSTRACT

The present invention provides methods for diagnosing cancers, such as prostate cancer. Also, methods for evaluating the prostate cancer state of a patient are described herein. These methods involve the detection, analysis, and classification of biological patterns in biological samples. The biological patterns are obtained using, for example, mass spectrometry systems, antibody based techniques, or nucleic acid based techniques. The present invention also includes therapeutic and prophylactic agents that target the biomarkers described herein. Also, the present invention provides methods for the treatment of prostate cancer using the markers described herein or agents that mimic the properties of these markers.

### BACKGROUND OF THE INVENTION

Cancers are a complex set of diseases that result from genetic alterations both inherited and accrued over the lifetime of the individual. These genetic changes give rise to molecular alterations that distinguish cancer cells from normal cells. The number and type of alterations underlying cancers vary not only between cancers but also over the progression of the cancer and even within individual cancers. This results in an enormous diversity of phenotypes, especially at the molecular level, and corresponds with the observed diversity in path of progression, outcome, and response to therapy of various cancers, even when they have common presentation.

The current inability to distinguish between cancers, or to predict their prognosis and likely response to treatment, is a result of the inability to adequately identify and assess the biological state of an individual. This is reflected in the limited ability to detect the earliest stages of disease (e.g. stage I cancer detection), anticipate the path any apparent disease will take in one patient versus another (e.g. metastasis or remission prediction), predict the likelihood of response for any individual to a particular treatment (e.g. adjuvant and neo-adjuvant chemotherapeutic responses), and preempt the possible adverse effects of treatments on a particular individual (e.g. monitoring toxicology due to chemotherapy). New technologies and strategies are needed to define biological states related to cancer and thereby inform medical care and improve the repertoire of medical tools to treat cancer patients.

### BRIEF SUMMARY OF THE INVENTION

One aspect of the present invention provides methods for the diagnosis of cancer, such as prostate cancer. In one embodiment, prostate cancer states are analyzed using the prostate cancer markers described herein. These markers can be detected using mass spectrometry, antibody based techniques, nucleic acid based techniques, or any other suitable technique known in the art.

Another aspect of the invention includes prostate cancer therapeutic agents that modulate the markers described herein. In one embodiment, the markers themselves or agents that mimic their properties are used in the treatment of prostate cancer.

One aspect of the invention is a method for diagnosing prostate cancer comprising identifying one or two prostrate cancer markers in a biological sample, the markers being those markers that can provide mass spectral signals selected from following approximate m/z values:

| Biomarker | |
|---|---|
| (*molecular weight for the indicated | |
| entity is as shown or +1 dalton) | Observed m/z (thomson) |
| 1* | 2.9511E+02 |
| 2 | 1.5433E+03 |
| | 1.3890E+03 |
| | 1.2629E+03 |
| | 1.1577E+03 |
| | 1.0687E+03 |
| | 9.9246E+02 |
| | 9.2636E+02 |
| | 8.6852E+02 |
| | 8.1749E+02 |
| | 7.7213E+02 |
| | 7.3155E+02 |
| | 6.9502E+02 |
| | 6.6197E+02 |

and providing a prostrate cancer diagnosis based on a review of the subset of prostate cancer markers. In some embodiments, the second step of providing a diagnosis is an optional step. In another embodiment, the markers are further characterized by the following approximate molecular weights and charge states:

| Biomarker | | | |
|---|---|---|---|
| (*molecular weight for the | | | Molecular |
| indicated monoisotopic entities is | | Observed m/z | Weight |
| as shown or +1 dalton) | Charge | (thomson) | (Daltons) |
| 1* | 1 | 2.9511E+02 | 294 |
| 2 | 9 | 1.5433E+03 | 13880 |
| | 10 | 1.3890E+03 | 13880 |
| | 11 | 1.2629E+03 | 13880 |
| | 12 | 1.1577E+03 | 13880 |
| | 13 | 1.0687E+03 | 13880 |
| | 14 | 9.9246E+02 | 13880 |
| | 15 | 9.2636E+02 | 13880 |
| | 16 | 8.6852E+02 | 13880 |
| | 17 | 8.1749E+02 | 13880 |
| | 18 | 7.7213E+02 | 13880 |
| | 19 | 7.3155E+02 | 13880 |
| | 20 | 6.9502E+02 | 13880 |
| | 21 | 6.6197E+02 | 13880 |

In another embodiment, the method of diagnosis further comprises identifying additional prostate cancer markers in a biological sample such as prostate specific antigen, human glandular kallikerin 2, prostatic acid phosphatase, prostate-specific membrane antigen, androgen receptor, insulin-like growth factor, and/or insulin-like growth factor binding protein.

Another aspect of the invention is a method for diagnosing a patient by identifying one or more of the following prostate cancer markers in a biological sample, these markers being characterized by following approximate molecular weights:

| Biomarker | |
|---|---|
| (*molecular | |
| weight for the | |
| indicated entities | |
| is as shown or +1 | Molecular Weight |
| dalton) | (Daltons) |
| 1* | 294 |
| 2 | 13880 |
| 3 | 1050 |
| 4 | 519 |
| 5 | 9061 |
| 6 | 4201 |
| 7* | 496 |
| 8 | 3331 |
| 9 | 2162 |
| 10 | 6169 |
| 11 | 3307 |
| 12 | 9288 |
| 13 | 7728 |
| 14 | 9289 |
| 15 | 3224 |
| 16 | 764 |
| 17* | 618 |
| 18 | 5720 |
| 19 | 1397 |
| 20 | 11439 |
| 21 | 14043 |
| 22 | 1626 |
| 23* | 333 |
| 24 | 13727 |
| 25 | 13876 |
| 26* | 228 |
| 27* | 326 |
| 28 | 965 |
| 29* | 256 |
| 30 | 624 |
| 31 | 894 |
| 32 | 856 |
| 33 | 12451 |
| 34 | 1855 |
| 35 | 11729 |
| 36 | 13897 |
| 37 | 13841 |
| 38 | 13978 |
| 39 | 6630 |
| 40* | 686 |
| 41* | 312 |
| 42 | 1465 |
| 43 | 981 |
| 44 | 943 |
| 45* | 272 |
| 46* | 228 |
| 47* | 341 |

and providing a prostrate cancer diagnosis based on a review of the levels of these prostate cancer markers. In some embodiments, the second step of providing a diagnosis is an optional step.

In some embodiments, the markers are further characterized by following charge states and/or approximate m/z ratios:

| Biomarker | | | |
|---|---|---|---|
| (*molecular | | | |
| weight for the | | | |
| indicated | | | |
| monoisotopic | | | |
| entities is as | | | Molecular |
| shown or +1 | | Observed m/z | Weight |
| dalton) | Charge | (thomson) | (Daltons) |
| 3 | 2 | 5.2576E+02 | 1050 |
| 4 | 1 | 5.2035E+02 | 519 |
| | 2 | 2.6067E+02 | 519 |
| 5 | 8 | 1.1336E+03 | 9061 |
| | 9 | 1.0077E+03 | 9061 |
| | 10 | 9.0707E+02 | 9061 |
| 6 | 4 | 1.0513E+03 | 4201 |
| | 5 | 8.4127E+02 | 4201 |
| 7* | 1 | 4.9723E+02 | 496 |
| 8 | 3 | 1.1113E+03 | 3331 |
| | 4 | 8.3369E+02 | 3331 |
| | 5 | 6.6715E+02 | 3331 |
| 9 | 3 | 7.2164E+02 | 2162 |
| | 4 | 5.4148E+02 | 2162 |
| 10 | 6 | 1.0291E+03 | 6169 |
| | 7 | 8.8222E+02 | 6169 |
| | 8 | 7.7207E+02 | 6169 |
| 11 | 4 | 8.2773E+02 | 3307 |
| 12 | 7 | 1.3279E+03 | 9288 |
| | 8 | 1.1620E+03 | 9288 |
| | 9 | 1.0330E+03 | 9288 |
| | 10 | 9.2982E+02 | 9288 |
| 13 | 7 | 1.1050E+03 | 7728 |
| | 8 | 9.6701E+02 | 7728 |
| | 9 | 8.5967E+02 | 7728 |
| 14 | 7 | 1.3279E+03 | 9289 |
| | 8 | 1.1621E+03 | 9289 |
| | 9 | 1.0331E+03 | 9289 |
| | 10 | 9.2986E+02 | 9289 |
| 15 | 4 | 8.0696E+02 | 3224 |
| | 5 | 6.4576E+02 | 3224 |
| 16 | 1 | 7.6536E+02 | 764 |
| | 2 | 3.8318E+02 | 764 |
| 17* | 1 | 6.1935E+02 | 618 |
| 18 | 6 | 9.5430E+02 | 5720 |
| | 7 | 8.1812E+02 | 5720 |
| | 8 | 7.1598E+02 | 5720 |
| | 9 | 6.3653E+02 | 5720 |
| 19 | 2 | 6.9929E+02 | 1397 |
| 20 | 12 | 9.5422E+02 | 11439 |
| | 13 | 8.8089E+02 | 11439 |
| | 14 | 8.1804E+02 | 11439 |
| | 15 | 7.6357E+02 | 11439 |
| | 16 | 7.1591E+02 | 11439 |
| | 17 | 6.7386E+02 | 11439 |
| | 18 | 6.3648E+02 | 11439 |
| 21 | 13 | 1.0812E+03 | 14043 |
| | 14 | 1.0040E+03 | 14043 |
| | 15 | 9.3718E+02 | 14043 |
| | 16 | 8.7867E+02 | 14043 |
| | 17 | 8.2704E+02 | 14043 |
| | 18 | 7.8115E+02 | 14043 |
| | 19 | 7.4009E+02 | 14043 |
| 22 | 3 | 5.4295E+02 | 1626 |
| | 4 | 4.0747E+02 | 1626 |
| 23* | 1 | 3.3413E+02 | 333 |
| 24 | 13 | 1.0569E+03 | 13727 |
| | 14 | 9.8152E+02 | 13727 |
| | 15 | 9.1615E+02 | 13727 |
| | 16 | 8.5896E+02 | 13727 |
| | 17 | 8.0849E+02 | 13727 |
| | 18 | 7.6363E+02 | 13727 |
| | 19 | 7.2349E+02 | 13727 |
| 25 | 14 | 9.9214E+02 | 13876 |
| | 15 | 9.2607E+02 | 13876 |
| | 16 | 8.6825E+02 | 13876 |
| | 17 | 8.1723E+02 | 13876 |
| | 18 | 7.7189E+02 | 13876 |
| 26* | 1 | 2.2911E+02 | 228 |
| 27* | 1 | 3.2712E+02 | 326 |
| 28 | 2 | 4.8368E+02 | 965 |
| 29* | 1 | 2.5715E+02 | 256 |
| 30 | 1 | 6.2533E+02 | 624 |
| | 2 | 3.1316E+02 | 624 |
| | 3 | 2.0911E+02 | 624 |
| 31 | 2 | 4.4813E+02 | 894 |
| 32 | 1 | 8.5739E+02 | 856 |
| | 2 | 4.2920E+02 | 856 |
| 33 | 7 | 1.7797E+03 | 12451 |
| | 8 | 1.5574E+03 | 12451 |
| | 9 | 1.3845E+03 | 12451 |
| 34 | 3 | 6.1932E+02 | 1855 |
| 35 | 10 | 1.1739E+03 | 11729 |
| | 11 | 1.0673E+03 | 11729 |
| | 12 | 9.7840E+02 | 11729 |
| | 13 | 9.0322E+02 | 11729 |
| | 14 | 8.3878E+02 | 11729 |
| 36 | 13 | 1.0700E+03 | 13897 |
| | 14 | 9.9366E+02 | 13897 |
| | 15 | 9.2748E+02 | 13897 |
| | 16 | 8.6957E+02 | 13897 |
| | 17 | 8.1848E+02 | 13897 |
| | 18 | 7.7307E+02 | 13897 |
| | 19 | 7.3243E+02 | 13897 |
| | 20 | 6.9586E+02 | 13897 |
| 37 | 11 | 1.2593E+03 | 13841 |
| | 12 | 1.1544E+03 | 13841 |
| | 13 | 1.0657E+03 | 13841 |
| | 14 | 9.8967E+02 | 13841 |
| | 15 | 9.2376E+02 | 13841 |
| | 16 | 8.6609E+02 | 13841 |
| | 17 | 8.1520E+02 | 13841 |
| | 18 | 7.6997E+02 | 13841 |
| | 19 | 7.2949E+02 | 13841 |
| 38 | 11 | 1.2717E+03 | 13978 |
| | 12 | 1.1659E+03 | 13978 |
| | 13 | 1.0762E+03 | 13978 |
| | 14 | 9.9944E+02 | 13978 |
| | 15 | 9.3288E+02 | 13978 |
| | 16 | 8.7464E+02 | 13978 |
| | 17 | 8.2325E+02 | 13978 |
| | 18 | 7.7757E+02 | 13978 |
| 39 | 6 | 1.1060E+02 | 6630 |
| | 7 | 9.4818E+02 | 6630 |
| | 8 | 8.2978E+02 | 6630 |
| | 9 | 7.3769E+02 | 6630 |
| | 10 | 6.6402E+02 | 6630 |
| | 11 | 6.0375E+02 | 6630 |
| 40* | 1 | 6.8650E+02 | 686 |
| 41* | 1 | 3.1314E+02 | 312 |
| 42 | 2 | 7.3335E+02 | 1465 |
| | 3 | 4.8924E+02 | 1465 |
| | 4 | 3.6718E+02 | 1465 |
| 43 | 2 | 4.9167E+02 | 981 |
| 44 | 1 | 9.4442E+02 | 943 |
| | 2 | 4.7271E+02 | 943 |
| 45* | 1 | 2.7310E+02 | 272 |
| 46* | 1 | 2.2911E+02 | 228 |
| 47* | 1 | 3.4215E+02 | 341 |

In another embodiment, the method of diagnosis further comprises identifying additional prostate cancer markers in a biological sample such as prostate specific antigen, human glandular kallikrein 2, prostatic acid phosphatase, prostate-specific membrane antigen, androgen receptor, insulin-like growth factor, and/or insulin-like growth factor binding protein.

Another aspect of the invention is a method of analyzing prostate cancer states comprising identifying the presence of transthyretin, fragments of transthyretin, and/or post-translationally modified forms of transthyretin in a biological sample and making a decision regarding a prostate cancer state, wherein said biological sample is obtained from a subject with PSA levels of less than about 6 ng/ml.

Preferably the methods of the present invention are performed using a mass spectrometry system, such as a time-of-flight mass spectrometry system. In preferred embodiments, the said biological sample is delivered to the mass spectrometry system by electrospray ionization or by matrix assisted laser desorption ionization. The biological sample can be separated using microchannel electrophoresis or capillary electrophoresis on a chip format. The sample can be prepared and/or separated on a microfluidics device. The markers can also be identified using an antibody-based technique, a multiplexed antibody array, a multiplexed antibody bead, a protein affinity chip, an aptamer, and/or a microsequencing technique.

In one aspect, the invention is a diagnostic product for prostate cancer with at least one component adapted and configured for identifying and/or analyzing the following biomarkers:

| Biomarker | |
|---|---|
| (*molecular | |
| weight for the | |
| indicated entities | |
| is as shown or +1 | Molecular Weight |
| dalton) | (Daltons) |
| 1* | 294 |
| 2 | 13880 |
| 3 | 1050 |
| 4 | 519 |
| 5 | 9061 |
| 6 | 4201 |
| 7* | 496 |
| 8 | 3331 |
| 9 | 2162 |
| 10 | 6169 |
| 11 | 3307 |
| 12 | 9288 |
| 13 | 7728 |
| 14 | 9289 |
| 15 | 3224 |
| 16 | 764 |
| 17* | 618 |
| 18 | 5720 |
| 19 | 1397 |
| 20 | 11439 |
| 21 | 14043 |
| 22 | 1626 |
| 23* | 333 |
| 24 | 13727 |
| 25 | 13876 |
| 26* | 228 |
| 27* | 326 |
| 28 | 965 |
| 29* | 256 |
| 30 | 624 |
| 31 | 894 |
| 32 | 856 |
| 33 | 12451 |
| 34 | 1855 |
| 35 | 11729 |
| 36 | 13897 |
| 37 | 13841 |
| 38 | 13978 |
| 39 | 6630 |
| 40* | 686 |
| 41* | 312 |
| 42 | 1465 |
| 43 | 981 |
| 44 | 943 |
| 45* | 272 |
| 46* | 228 |
| 47* | 341 |

Another aspect of the invention is a method of diagnosing prostate cancer by reviewing a pattern of prostrate cancer markers from the subject, the pattern being one or both of the following markers which are characterized with the following approximate m/z values:

| Biomarker | |
|---|---|
| (*molecular weight | |
| for the indicated | |
| entity is as shown or | Observed m/z |
| +1 dalton) | (thomson) |
| 1* | 2.9511E+02 |
| 2 | 1.5433E+03 |
| | 1.3890E+03 |
| | 1.2629E+03 |
| | 1.1577E+03 |
| | 1.0687E+03 |
| | 9.9246E+02 |
| | 9.2636E+02 |
| | 8.6852E+02 |
| | 8.1749E+02 |
| | 7.7213E+02 |
| | 7.3155E+02 |
| | 6.9502E+02 |
| | 6.6197E+02 |

and providing a prostrate cancer diagnosis to a patient, a health care provider or a health care manager based on the review of the marker pattern. In one embodiment, the markers are further characterized by following approximate molecular weights and charge states:

| Biomarker | | | |
|---|---|---|---|
| (*molecular weight for the | | | Molecular |
| indicated monoisotopic entities is | | Observed m/z | Weight |
| as shown or +1 dalton) | Charge | (thomson) | (Daltons) |
| 1* | 1 | 2.9511E+02 | 294 |
| 2 | 9 | 1.5433E+03 | 13880 |
| | 10 | 1.3890E+03 | 13880 |
| | 11 | 1.2629E+03 | 13880 |
| | 12 | 1.1577E+03 | 13880 |
| | 13 | 1.0687E+03 | 13880 |
| | 14 | 9.9246E+02 | 13880 |
| | 15 | 9.2636E+02 | 13880 |
| | 16 | 8.6852E+02 | 13880 |
| | 17 | 8.1749E+02 | 13880 |
| | 18 | 7.7213E+02 | 13880 |
| | 19 | 7.3155E+02 | 13880 |
| | 20 | 6.9502E+02 | 13880 |
| | 21 | 6.6197E+02 | 13880 |

One aspect is a method for diagnosis of prostate cancer by identifying one or more of the following prostate cancer markers with mass spectroscopy where the markers are characterized by the following approximate molecular weights:

| Biomarker | |
|---|---|
| (*molecular | |
| weight for the | |
| indicated entities | |
| is as shown or +1 | Molecular Weight |
| dalton) | (Daltons) |
| 1* | 294 |
| 2 | 13880 |
| 3 | 1050 |
| 4 | 519 |
| 5 | 9061 |
| 6 | 4201 |
| 7* | 496 |
| 8 | 3331 |
| 9 | 2162 |
| 10 | 6169 |
| 11 | 3307 |
| 12 | 9288 |
| 13 | 7728 |
| 14 | 9289 |
| 15 | 3224 |
| 16 | 764 |
| 17* | 618 |
| 18 | 5720 |
| 19 | 1397 |
| 20 | 11439 |
| 21 | 14043 |
| 22 | 1626 |
| 23* | 333 |
| 24 | 13727 |
| 25 | 13876 |
| 26* | 228 |
| 27* | 326 |
| 28 | 965 |
| 29* | 256 |
| 30 | 624 |
| 31 | 894 |
| 32 | 856 |
| 33 | 12451 |
| 34 | 1855 |
| 35 | 11729 |
| 36 | 13897 |
| 37 | 13841 |
| 38 | 13978 |
| 39 | 6630 |
| 40* | 686 |
| 41* | 312 |
| 42 | 1465 |
| 43 | 981 |
| 44 | 943 |
| 45* | 272 |
| 46* | 228 |
| 47* | 341 |

and providing a prostate cancer diagnosis based a review of the identified prostate cancer markers. In some embodiments, the second step of providing a diagnosis is an optional step.

The invention also includes a computer-readable medium suitable for transmission of the result of an analysis of a biological sample, this result being information regarding the presence and/or levels or one or more biomarkers 1-47. The medium could also contain information regarding the prostate cancer diagnosis of a subject based on the presence and/or levels or one or more biomarkers 1-47.

One aspect of the invention is the use of at least one of the following biomarkers:

| Biomarker | |
|---|---|
| (*molecular | |
| weight for the | |
| indicated entities | |
| is as shown or +1 | Molecular Weight |
| dalton) | (Daltons) |
| 1* | 294 |
| 2 | 13880 |
| 3 | 1050 |
| 4 | 519 |
| 5 | 9061 |
| 6 | 4201 |
| 7* | 496 |
| 8 | 3331 |
| 9 | 2162 |
| 10 | 6169 |
| 11 | 3307 |
| 12 | 9288 |
| 13 | 7728 |
| 14 | 9289 |
| 15 | 3224 |
| 16 | 764 |
| 17* | 618 |
| 18 | 5720 |
| 19 | 1397 |
| 20 | 11439 |
| 21 | 14043 |
| 22 | 1626 |
| 23* | 333 |
| 24 | 13727 |
| 25 | 13876 |
| 26* | 228 |
| 27* | 326 |
| 28 | 965 |
| 29* | 256 |
| 30 | 624 |
| 31 | 894 |
| 32 | 856 |
| 33 | 12451 |
| 34 | 1855 |
| 35 | 11729 |
| 36 | 13897 |
| 37 | 13841 |
| 38 | 13978 |
| 39 | 6630 |
| 40* | 686 |
| 41* | 312 |
| 42 | 1465 |
| 43 | 981 |
| 44 | 943 |
| 45* | 272 |
| 46* | 228 |
| 47* | 341 |

in a method for the identification of a prostatic cancer state in a subject. Preferably, the use employs mass spectroscopic determination of the markers.

One aspect of the invention is the use of transthyretin, a fragment thereof and/or post-translationally modified forms of transthyretin in a method for the identification of a prostatic cancer state in a subject with a serum PSA level of less than 6 ng/ml. One embodiment is the use of transthyretin with a molecular weight of 13,880. Preferably, the use employs mass spectroscopic determination of transthyretin, a fragment thereof and/or post-translationally modified forms of transthyretin and optionally a compound of molecular weight 294. Preferably, the amount of the defined agents are compared to the amount in a sample from a person not having prostate cancer.

### BRIEF DESCRIPTION OF THE FIGURES

FIG. 1 is a schematic representation of the experimental design.

FIG. 2 is a schematic representation of an embodiment of the sample preparation process.

FIG. 3 is a flowchart illustrating an embodiment of a method of the invention.

FIG. 4 is a flowchart illustrating an embodiment of a method of the invention.

FIG. 5 depicts an apparatus suitable for use in the methods of the invention.

FIG. 6 illustrates an apparatus suitable for use in the methods of the invention.

FIG. 7 is a flowchart illustrating an embodiment of a method of the invention.

### DETAILED DESCRIPTION OF THE INVENTION

In one aspect, the present invention provides methods for diagnosing prostate cancer. Also, methods for evaluating the prostate cancer state of a patient are described herein. These methods involve the detection, analysis, and classification of biological markers in biological samples. It is possible to detect prostate cancer using one of the identified markers of the invention but in a preferred embodiment, a pattern of markers allows detection, diagnosis and prognosis of prostate cancer. Biological patterns are typically composed of signals from markers such as, but not limited to, proteins, peptides, protein fragments, small molecules, sugars, lipids, fatty acids, or any other component found in a biological sample. The term “protein” as used herein refers to an organic compound comprising two or more amino acids covalently joined by peptide bonds. Proteins include, but are not limited to, peptides, oligopeptides, glycosylated peptides, and polypeptides. The biological patterns used in the present invention are typically patterns of markers. Preferably, the markers identified and used in the present invention are prostate cancer markers. The terms “markers” and “biomarkers” are used herein interchangeably. It is preferred that the biomarkers comprise one or more proteins. The method comprises detecting one or more biomarker and preferably detecting a pattern of biomarkers. Preferably the number of markers in these patterns can be more than about 5, more preferably more than about 25, even more preferably more than about 45, and even more preferably more than about 100. In some embodiments, the markers being analyzed do not include glycolipids or oligosaccharides.

The markers may be detected using any suitable conventional analytical technique including but not limited to, immunoassays, protein chips, multiplexed immunoassays, complex detection with aptamers, chromatographic separation with spectrophotometric detection and preferably mass spectroscopy. It is preferred when identifying—biological patterns—that the analysis use—mass spectrometry systems. Embodiments may or may not involve the use of protein affinity chips, for example chips with specific or non-specific binding surfaces (e.g. hydrophobic surfaces). In some embodiments, the samples are prepared and separated with fluidic devices, preferably microfluidic devices, and delivered to the mass spectrometry system by electrospray ionization (ESI). In some embodiments, the delivery happens “on-line”, e.g. the separations device is directly interfaced to a mass spectrometer and the spectra are collected as fractions move from the column, through the ESI interface into the mass spectrometer. In other embodiments, fractions are collected from the separations device (e.g. “off-line”) and those fractions are later run using direct-infusion ESI mass spectrometry. In yet another embodiment, the samples are prepared and separated with fluidic devices, preferably microfluidic devices, and spotted on a MALDI plate for laser-desorption ionization.

The identification and analysis of cancer markers, especially prostate cancer markers, have numerous therapeutic and diagnostic purposes. Clinical applications include, for example, detection of disease; distinguishing disease states to inform prognosis, selection of therapy, and/or prediction of therapeutic response; disease staging; identification of disease processes; prediction of efficacy of therapy; monitoring of patients trajectories (e.g., prior to onset of disease); prediction of adverse response; monitoring of therapy associated efficacy and toxicity; prediction of probability of occurrence; recommendation for prophylactic measures; and detection of recurrence. Also, these cancer markers can be used in assays to identify novel therapeutics. In addition, the markers can be used as targets for cancer drugs, especially prostate cancer drugs, and therapeutics, for example antibodies against the markers or fragments of the markers can be used as prostate cancer therapeutics. The present invention also includes therapeutic and prophylactic agents that target the biomarkers described herein. In addition, the markers can be used as prostate cancer drugs or therapeutics themselves.

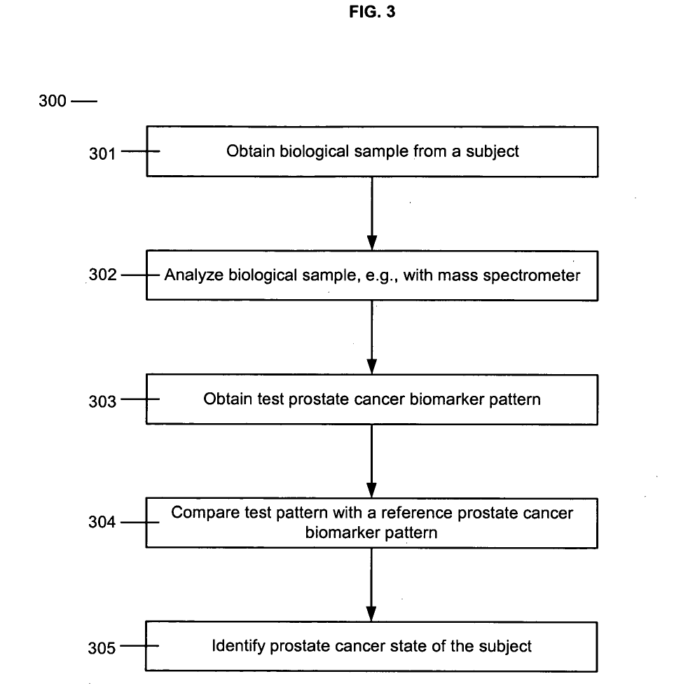

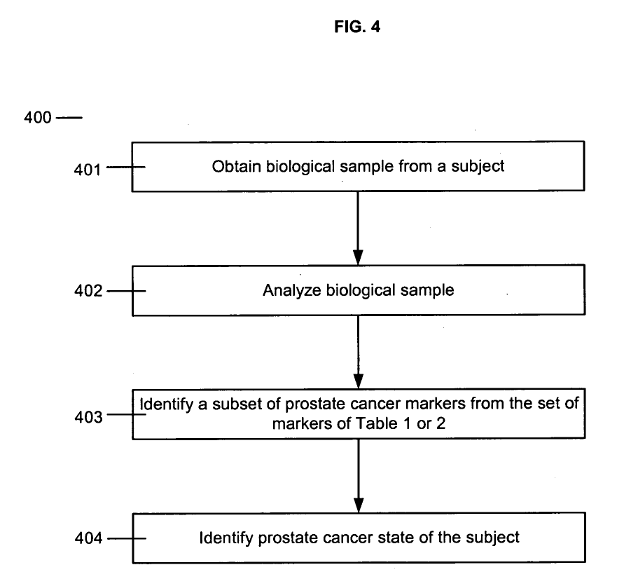

The method typically involves sample preparation, sample separation and detection or analysis of one or more biomarkers. Two embodiments of the methods of the present invention are depicted in FIGS. 3 and 4. In—FIG. 3, a biological sample is obtained from a subject, preferably a human, at step 301. The sample is analyzed with a mass spectrometer at step 302. A test biomarker pattern is obtained for the subject at step 303 and this test pattern is compared with a reference pattern at step 304. Based on this comparison a decision is made regarding the cancer state, such as the prostate cancer state, of the subject. Preferably, the test and reference patterns are protein patterns or protein patterns in combination with other cellular components. The reference pattern may be obtained from the same subject or from a different subject who is either not affected with the disease or is a prostate cancer patient. The reference pattern could be obtained from one subject or multiple subjects. In—FIG. 4, a biological sample is obtained from a subject at step 401. The biological sample is analyzed at step 402 and the analysis is conducted using a technique suitable for identifying one or more cancer markers of Table 1 and/or Table 2. The prostate cancer markers are identified at step 403 and based on this identification a decision is made regarding the prostate cancer state of the subject at step 404.

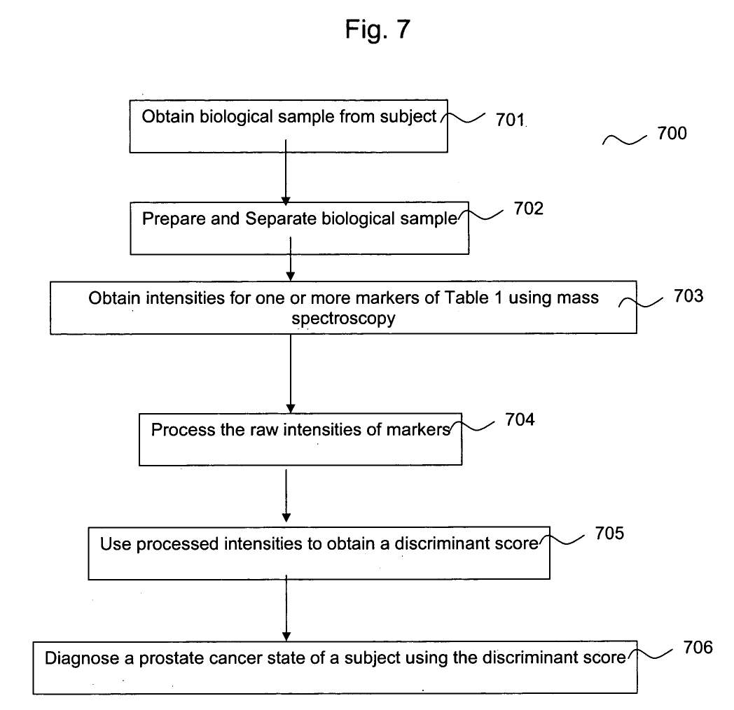

In one method of the invention, as shown in FIG. 7, a biological sample is obtained from a subject at step 701, the biological sample is prepared and separated at step 702, intensities for one or more markers of Table 1 are obtained using mass spectroscopy at step 703, the raw intensities of the markers are processed at step 704, the processed intensities are used to obtain a discriminant score at step 705, and the discriminant score is used to diagnose a prostate cancer state of a subject at step 706.

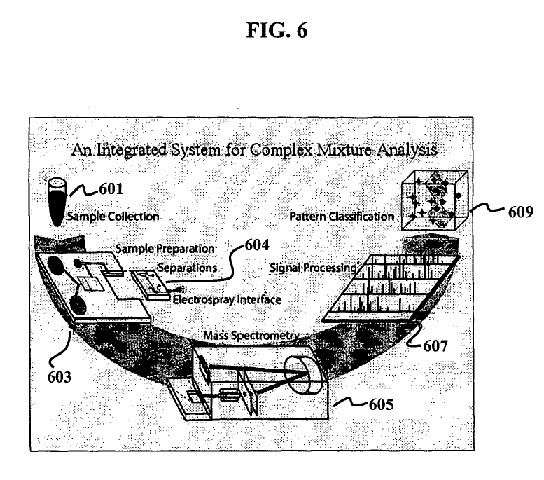

FIG. 6 illustrates an exemplary system platform suitable for use herein. The sample tested could be a biological fluid or tissue or cells. Biological fluids 601 include but are not limited to serum, plasma, whole blood, nipple aspirate, pancreatic fluid, trabecular fluid, lung lavage, urine, cerebrospinal fluid, saliva, sweat, pericrevicular fluid, semen, prostatic fluid, pre-ejaculate fluid, nasal discharge, and tears. The system provides for the integration of fast molecular separations and electrospray ionization system 604 on a microfluidics platform 603. The system provides processed samples to a high sensitivity time of flight mass spectrometer 605. Signal processing system and pattern extraction and recognition tools 605 incorporate domain knowledge to extract information from biomarker patterns and classify the patterns to provide a classification 609. The microfluidics device(s) 603 may be formed in plastic by means of etching, machining, cutting, molding, casting or embossing. The microfluidics device(s) for sample preparation and separation may be made from glass or silicon by means of etching, machining, or cutting. The device may be formed by polymerization on a form or other mold. The molecular separations unit or the integrated fast molecular separations/electrospray ionization unit may provide additional sample preparation steps, including sample loading, sample concentration, removal of salts and other compounds that may interfere with electrospray ionization, removal of highly abundant species, proteolytic or chemical cleavage of components within the biological material, and/or aliquoting in to storage containers.

One embodiment of the invention is a method for detection and diagnosis of cancer comprising detecting at least one or more biomarkers in a subject sample, and correlating the detection of one or more biomarkers with a diagnosis of cancer, wherein the correlation takes into account the detection of one or more biomarker in each diagnosis, as compared to normal subjects, wherein the biomarkers are selected from biomarkers 1-45 depicted in Table 1 below. In preferred methods, the step of correlating the measurement of the biomarkers with cancer status is performed by a software algorithm. Preferably, the data generated is transformed into computer readable form; and an algorithm is executed that classifies the data according to user input parameters, for detecting signals that represent markers present in cancer patients and are lacking or present at different levels in non-cancer subjects.

Purified markers for screening and aiding in the diagnosis of cancer and/or generation of antibodies for further diagnostic assays are provided for. Purified markers are selected from the biomarkers of Table 1.

The invention further provides for kits for aiding the diagnosis of cancer, comprising at least one agent to detect the presence of one or more biomarkers, wherein the agent detects one or more biomarker selected from the biomarkers of Table 1. Preferably, the kit comprises written instructions for use of the kit for detection of cancer and the instructions provide for contacting a test sample with the agent and detecting one or more biomarkers retained by the agent. A kit for diagnosis could also include a computer readable medium with information regarding the patterns of biomarkers in normal and/or prostate cancer patients with or without instructions for the use of the information on the computer readable medium to diagnose prostate cancer.

Methods and Systems for Determining Patterns of Cancer Markers

Collection, Preparation, and Separation of Biological Sample

Biological samples are obtained from individuals with varying phenotypic states, particularly various states of prostate cancer. Examples of phenotypic states also include phenotypes of a non-cancerous state, which is typically used for comparisons to prostate cancer states. Other examples of phenotypic states include other prostate diseases or other cancers. In a preferred embodiment, examples of various phenotypic states of prostate cancer are matched with control samples that are obtained from individuals who do not exhibit the phenotypic state of prostate cancer (e.g., an individual who is not affected by a disease).

Samples may be collected from a variety of sources in a given patient. Samples collected are preferably bodily fluids such as blood, serum, sputum, including, saliva, plasma, nipple aspirants, synovial fluids, cerebrospinal fluids, sweat, urine, fecal matter, pancreatic fluid, trabecular fluid, cerebrospinal fluid, tears, bronchial lavage, swabbings, bronchial aspirants, semen, prostatic fluid, precervicular fluid, vaginal fluids, pre-ejaculate, etc. In a preferred embodiment, a sample collected is approximately 1 to approximately 5 ml of blood. In another preferred embodiment, a sample collected is approximately 10 to approximately 15 ml of blood.

In some instances, samples may be collected from individuals repeatedly over a longitudinal period of time (e.g., about once a day, once a week, once a month, biannually or annually). Obtaining numerous samples from an individual over a period of time can be used to verify results from earlier detections and/or to identify an alteration in biological pattern as a result of, for example, disease progression, drug treatment, etc. Samples can be obtained from humans or non-humans. In a preferred embodiment, samples are obtained from humans. In a preferred embodiment, serum is derived from collected blood and then analyzed. Preferably, blood is processed into serum and frozen at e.g., −80° C. until further use within 6 hours.

Sample preparation and separation can involve any of the following procedures, depending on the type of sample collected and/or types of biological molecules searched: concentration, dilution, adjustment of pH, removal of high abundance polypeptiaes (e.g., albumin, gamma globulin, and transferin, etc.); addition of preservatives and calibrants, addition of protease inhibitors, addition of denaturants, desalting of samples; concentration of sample proteins; protein digestions; and fraction collection. The sample preparation can also isolate molecules that are bound in non-covalent complexes to other protein (e.g., carrier proteins). This process may isolate only those molecules bound to a specific carrier protein (e.g., albumin), or use a more general process, such as the release of bound molecules from all carrier proteins via protein denaturation, for example using an acid, followed by removal of the carrier proteins. Preferably, sample preparation-techniques concentrate information-rich proteins (e.g., proteins that have “leaked” from diseased cells) and deplete proteins that would carry little or no information such as those that are highly abundant or native to serum

Sample preparation can take place in a multiplicity of devices including preparation and separation devices or on a combination separation device. In a preferred embodiment, such separation device is a microfluidics device. Optimally, the separation device interfaces directly or indirectly with a detection device. In another embodiment, such separation device is a fluidics device.

Approximately 1 μL, 10 μL, 100 μL, or 1000 μL of a sample is analyzed per assay in some embodiments of the invention. Removal of undesired proteins (e.g., high abundance, uninformative, or undetectable proteins) can be achieved using high affinity reagents, high molecular weight filters, ultracentrifugation and/or electrodialysis. High affinity reagents include antibodies or other reagents (e.g. aptamers) that selectively bind to high abundance proteins. Sample preparation could also include ion exchange chromatography, metal ion affinity chromatography, gel filtration, hydrophobic chromatography, chromatofocusing, adsorption chromatography, isoelectric focusing and related techniques. Molecular weight filters include membranes that separate molecules on the basis of size and molecular weight. Such filters may further employ reverse osmosis, nanofiltration, ultrafiltration and microfiltration.

Ultracentriftigation is another method for removing undesired polypeptides. Ultracentrifugation is the centrifugation of a sample at about 60,000 rpm while monitoring with an optical system the sedimentation (or lack thereof) of particles. Finally, electrodialysis is a procedure which uses an electromembrane or semipermable membrane in a process in which ions are transported through semi-permeable membranes from one solution to another under the influence of a potential gradient. Since the membranes used in electrodialysis may have the ability to selectively transport ions having positive or negative charge and reject ions of the opposite charge, or to allow species to migrate through a semipermable membrane based on size and charge, electrodialysis is useful for concentration, removal, or separation of electrolytes.

In a preferred embodiment, the manifold or microfluidics device performs electrodialysis to remove high molecular weight polypeptides or undesired polypeptides. Electrodialysis is first used to allow only molecules under approximately e.g., 30 kD (not a sharp cutoff) to pass through into a second chamber. A second membrane with a very small molecular weight cut-off (e.g., roughly 500-1000 D) will allow smaller molecules to egress the second chamber.

After samples are prepared, components that may comprise a biological marker or pattern of interest may be separated. Separation can take place in the same location as the preparation or in another location. In a preferred embodiment, separation occurs in the same microfluidics device where preparation occurs, but in a different location on the device. Samples can be removed from an initial manifold location to a microfluidics device using various means, including an electric field. In a preferred embodiment, the samples are concentrated during their migration to the microfluidics device using reverse phase beads and an organic solvent elution such as about 50% methanol. This can elute the molecules into a channel or a well on a separation device of a microfluidics device.

Separation can involve any procedure known in the art, such as capillary electrophoresis (e.g., in capillary or on-chip) or chromatography (e.g., in capillary, column or on a chip).

Electrophoresis is a method which can be used to separate ionic molecules such as polypeptides according to their mobilities under the influence of an electric field. Electrophoresis can be conducted in a gel, capillary, or in a microchannel on a chip. In a capillary or microchannel, the mobility of a species is determined by the sum of the mobility of the bulk liquid in the capillary or microchannel, which can be zero or non-zero, and the electrophoretic mobility of the species, determined by the charge on the molecule and the frictional resistance the molecule encounters during migration. For molecules of regular geometry, the frictional resistance is often directly proportional to the size of the molecule, and hence it is common in the art for the statement to be made that molecules are separated by their charge and size. Examples of gels used for electrophoresis include starch, acrylamide, polyethylene oxides, agarose, or combinations thereof. In one embodiment, polyacrylamide gels are used. A gel can be modified by its cross-linking, addition of detergents, or denaturants, immobilization of enzymes or antibodies (affinity electrophoresis) or substrates (zymography) and incorporation of a pH gradient. Examples of capillaries used for electrophoresis include capillaries that interface with an electrospray.

Capillary electrophoresis (CE) is preferred for separating complex hydrophilic molecules and highly charged solutes. Advantages of CE include its use of small sample volumes (sizes ranging from 0.1 to 10 μl), fast separation, reproducibility, ease of automation, high resolution, and the ability to be coupled to a variety of detection methods, including mass spectrometry. CE technology, in general, relates to separation techniques that use narrow bore capillaries, commonly made of fused silica, to separate a complex array of large and small molecules. High voltages are used to separate molecules based on differences in charge, size and/or hydrophobicity. CE technology can also be implemented on microfluidic chips. Depending on the types of capillary and buffers used, CE can be further segmented into separation techniques such as capillary zone electrophoresis (CZE), capillary isoelectric focusing (CIEF), capillary isotachophoresis (cITP) and capillary electrochromatography (CEC). A preferred embodiment to couple CE techniques to electrospray ionization involves the use of volatile solutions, for example, aqueous mixtures containing a volatile acid and/or base and an organic such as an alcohol or acetonitrile.

Capillary isotachophoresis (cITP) is a technique in which the analytes move through the capillary at a constant speed but are nevertheless separated by their respective mobilities. This type of separation is accomplished in a heterogeneous buffer system where the buffers are different upstream and downstream of the sample zone. For a separation of positively-charged analytes, the buffer cation of the first buffer has a mobility and conductivity greater than that of the analytes, and the buffer cation of the second buffer has a mobility and conductivity less than that of the analytes. The voltage gradient per unit length of capillary depends on the conductivity, and therefore the voltage gradient is heterogeneous along the length of the capillary; higher in regions of low conductivity and lower in regions of high conductivity. At steady state, the analytes are focused in zones according to their mobility: if an analyte diffuses into a neighboring zone, it encounters a different field and will either speed up or slow down to rejoin its original zone. An advantage of cITP is that it can be used to concentrate a relatively wide zone of low concentration into a narrow zone of high concentration, thereby improving the limit of detection. Through the appropriate choice of buffers and injected zones, a hybrid separation technique often referred to as transient isotachophoresis-zone electrophoresis (tITP/ZE) can be performed. In tITP/ZE the conditions for isotachophoresis are present only transiently, after which the conditions are set up for zone electrophoresis. In this way, dilute samples can be concentrated and then separated into individual peaks.

Capillary zone electrophoresis (CZE), also known as free-solution CE (FSCE), is one of the simplest forms of CE. The separation mechanism of CZE is based on differences in the electrophoretic mobility of the species, determined by the charge on the molecule, and the frictional resistance the molecule encounters during migration which is often directly proportional to the size of the molecule. The separation typically relies on the charge state of the proteins, which is determined by the pH of the buffer solution.

Capillary isoelectric focusing (CIEF) allows weakly-ionizable amphoteric molecules, such as polypeptides, to be separated by electrophoresis in a pH gradient. A solute migrates to the point in the pH gradient where its net charge is zero. The pH of the solution at the point of zero net charge equals the isoelectric point (pI) of the solute. Because the solute is net neutral at the isoelectric point, its electrophoretic migration is no longer affected by the electric field, and the sample focuses into a tight zone. In CIEF, after all the solutes have focused at their pI's, the bulk solution is often moved past the detector by pressure or chemical means.

CEC is a hybrid technique between traditional liquid chromatography (HPLC) and CE. In essence, CE capillaries are packed with beads (as in traditional HPLC) or a monolith, and a voltage is applied across the packed capillary which generates an electro-osmotic flow (EOF). The EOF transports solutes along the capillary towards a detector. Both chromatographic and electrophoretic separation occurs during their transportation towards the detector. It is therefore possible to obtain unique separation selectivities using CEC compared to both HPLC and CE. The beneficial flow profile of EOF reduces flow related band broadening and separation efficiencies of several hundred thousand plates per meter are often obtained in CEC. CEC also makes it is possible to use small-diameter packings and achieve very high efficiencies.

Chromatography is another type of method for separating a subset of polypeptides, proteins, or other analytes. Chromatography can be based on the differential adsorption and elution of certain analytes or partitioning of analytes between mobile and stationary phases. Liquid chromatography (LC), for example, involves the use of fluid carrier over a non-mobile phase. Conventional analytical LC columns have an inner diameter of roughly 4.6 mm and a flow rate of roughly 1 ml/min. Micro-LC typically has an inner diameter of roughly 1.0 mm and a flow rate of roughly 40 μl/min. Capillary LC generally utilizes a capillary with an inner diameter of roughly 300 μm and a flow rate of approximately 5 μl/min. Nano-LC is available with an inner diameter of 50 μm-1 mm and flow rates of 200 μl/min. Nano-LC can vary in length (e.g., 5, 15, or 25 cm) and have typical packing of C18, 5 μm particle size. In a preferred embodiment, nano-LC is used. Nano-LC provides increased sensitivity due to lower dilution of chromatographic sample. The sensitivity improvement of nano-LC as compared to analytical HPLC is approximately 3700 fold.

In preferred embodiments, the samples are separated using capillary electrophoresis separation, more preferably CEC, or more preferably CZE. This will separate the molecules based on their electrophoretic mobility at a given pH and size (or hydrophobicity in the case of CEC).

In other preferred embodiments, the steps of sample preparation and separation are combined using microfluidics technology. A microfluidic device is a device that can transport fluids containing various reagents such as analytes and elutions between different locations using microchannel structures. Microfluidic devices provide advantageous miniaturization, automation and integration of a large number of different types of analytical operations. For example, continuous flow microfluidic devices have been developed that perform serial assays on extremely large numbers of different chemical compounds.

In a preferred embodiment, microfluidic devices are composed of plastic and formed by means of etching, machining, cutting, molding, casting or embossing. The microfluidics devices may alternatively be made from glass, silicon, or any other material by means of etching, machining, or cutting. The microfluidic devices may be either single use for a single sample; multi-use for a single sample at a time with serial loading; single use with parallel multiple sample processing; multi-use with parallel multiple sample processing; or a combination. Furthermore, more than one microfluidics device may be integrated into the system and can interface with a single detection device.

Once prepared and separated, the analytes are automatically delivered to a detection device, which detects the proteins or other analytes in a sample. In a preferred embodiment, proteins in elutions or solutions are delivered to a detection device by electrospray ionization (ESI). ESI operates by infusing a liquid containing the sample of interest through a channel or needle, which is kept at a potential of typically 1-6 kV, more typically of 1.5-4 kV. The voltage on the needle causes the spray to be charged as it is nebulized. The resultant charged vapor droplets disintegrate and evaporate in a region maintained between atmospheric pressure and a vacuum of several torr, until the solvent is essentially completely stripped off, leaving a charged ion. Alternatively, ions are formed by coulombic ejection from the surface of the droplet, in a process called ion evaporation. In either case, ions are then detected by a detection device such as a mass spectrometer. In a more preferred embodiment, nanospray ionization (NSI) is used. Nanospray ionization is a miniaturized version of ESI and provides low detection limits using extremely small volumes of sample fluid.

In preferred embodiments, separated proteins are directed down a channel that leads to an electrospray ionization emitter, which is built into a microfluidic device (an integrated ESI microfluidic device). Preferably, such an integrated ESI microfluidic device provides the detection device with samples at flow rates and complexity levels that are optimal for detection. Such flow rates are, preferably, approximately 10 μL/min—approximately 1000 μL/min, more preferably approximately 50 μL/min—approximately 200 μL/min. Furthermore, a microfluidic device is preferably aligned with a detection device for optimal sample capture. See co-pending U.S. application Ser. No. 10/681,742, filed on Jun. 12, 2003. For example, using dynamic feedback circuitry, a microfluidic device may allow for control positioning of an electrospray voltage and for the entire spray to be captured by the detection device orifice. The microfluidic device can be sold separately or in combination with other reagents, software tools and/or devices.

Calibrants can also be sprayed into detection device. Calibrants can be used to set instrument parameters and for signal processing purposes. Calibrants can be utilized before or in parallel with assessment of real sample. Calibrants can interface with a detection device using the same or a separate interface as the samples. In a preferred embodiment, calibrants are sprayed into a detection device using a second interface (e.g., second spray tip) or a second channel on the microfluidic device.

A biochip can also be used to separate the markers. Protein chips, also known as protein affinity chips, can be adapted for the capture of peptides and polypeptides. Many protein biochips are described in the art. These include, for example, protein biochips produced by Ciphergen Biosystems (Fremont, Calif.), Packard BioScience Company (Meriden Conn.), Zyomyx (Hayward, Calif.) and Phylos (Lexington, Mass.). Examples of such protein biochips are described in the following patents or patent applications: U.S. Pat. No. 6,225,047; International publication WO 99/51773; U.S. Pat. No. 6,329,209; International publication WO 00/66265; International publication WO 00/67293; U.S. publication 20030032043; and International publication WO 00/56934.

Additional suitable methods are disclosed in U.S. patent application entitled “SYSTEMS AND METHODS FOR DISCOVERY AND ANALYSIS OF MARKERS,” inventors Stults et al., attorney docket number “29191-707.502,” filed on Jul. 8, 2005, which is herein incorporated by reference.

Identification of Biological Patterns

Detection devices can comprise any suitable device that is able to detect proteins or other analytes presence and/or level, including for example, NMR, 2-D PAGE technology, Western blot technology, immuno-analysis technology, chromatography, or electrophoresis coupled to spectrophotometric detection either directly or after reaction of eluted products with a detection chemistry, and mass spectrometry. In some preferred embodiments, the methods herein rely on a mass spectrometer to detect marker patterns present in a given sample. There are various forms of mass spectrometers that may be utilized.

In certain embodiments, the methods utilize an ESI-MS detection device. An ESI-MS combines the ESI system with mass spectrometry. Furthermore, an ESI-MS preferably utilizes a time-of-flight (TOF) mass spectrometry system. In TOF-MS, ions are generated by whatever ionization method is being employed, such as ESI, and a voltage potential is applied. The potential extracts the ions from their source and accelerates them towards a detector. By measuring the time it takes the ions to travel a fixed distance, the mass to charge ratio of the ions can be calculated. TOF-MS can be set up to have an orthogonal-acceleration (OA). OA-TOF-MS are advantageous and preferred over conventional on-axis TOF because they have better spectral resolution and duty cycle. OA-TOF-MS also has the ability to obtain spectra, e.g., spectra of proteins and/or protein fragments, at a relatively high speed. In addition to the MS systems disclosed above, other forms of ESI-MS include quadrupole mass spectrometry, ion trap mass spectrometry, orbitrap mass spectrometry, Fourier transform ion cyclotron resonance (FTICR-MS), and hybrid combinations of these mass analyzers.

Quadrupole mass spectrometry consists of four parallel metal rods arranged in four quadrants (one rod in each quadrant). Two opposite rods have a positive applied potential and the other two rods have a negative potential. The applied voltages affect the trajectory of the ions traveling down the flight path. Only ions of a certain mass-to-charge ratio pass through the quadrupole filter and all other ions are thrown out of their original path. A mass spectrum is obtained by monitoring the ions passing through the quadrupole filter as the voltages on the rods are varied.

Ion trap mass spectrometry uses rf fields to trap ions. A quadrupole ion trap uses three electrodes in a small volume. The mass analyzer consists of a ring electrode separating two hemispherical electrodes. A linear ion trap uses end electrodes to trap ions in a linear quadrupole. A mass spectrum is obtained by changing the electrode voltages to eject the ions from the trap. The advantages of the ion-trap mass spectrometer include compact size, and the ability to trap and accumulate ions to increase the signal-to-noise ratio of a measurement.

Orbitrap mass spectrometry uses spatially defined electrodes with DC fields to trap ions. Ions are constrained by the DC field and undergo harmonic oscillation. The mass is determined based on the axial frequency of the ion in the trap. FTICR mass spectrometry is a mass spectrometric technique that is based upon an ion's motion in a magnetic field. Once an ion is formed, it eventually finds itself in the cell of the instrument, which is situated in a homogenous region of a large magnet. The ions are constrained in the XY plane by the magnetic field and undergo a circular orbit. The mass of the ion can be determined based on the cyclotron frequency of the ion in the cell.

In a preferred embodiment, the methods herein employ a TOF mass spectrometer, or more preferably, an ESI-TOF-MS, or more preferably an ESI-OA-TOF-MS or more preferably a mass spectrometer having a dual ion funnel to support dynamic switching between multiple quadrupoles in series, the second of which can be used to dynamically filter ions by mass in real time.

The detection device preferably interfaces with a separation/preparation device or microfluidic device, which allows for quick assaying of many of the proteins in a sample, or more preferably, most or all of the proteins in a sample. Preferably, a mass spectrometer is utilized that will accept a continuous sample stream for analysis and provide high sensitivity throughout the detection process (e.g., an ESI-MS). The separation/preparation device can also minimize ion suppression and therefore allow the detection of more proteins.

The detection system utilized preferably allows for the capture and measurement of most or all of the proteins that are introduced into the detection device. It is preferable that one can observe proteins with high information-content that are only present at low concentrations. By contrast, it is preferable to remove those polypeptide or components in advance that are, for example, common to all cells, especially those in high abundance or common in serum.

Immunoassays can be used to detect and analyze markers in a sample. Immunoassays typically comprise contacting a sample with an antibody that binds to a marker; and detecting the presence of a complex of the antibody bound to the marker in the sample. One aspect of the invention is a reagent with one or more purified marker 1-47 and/or antibodies against these markers and this reagent can be used in the diagnosis of prostate cancer. This reagent is preferably used to develop a reference marker pattern to which a marker pattern obtained from a test subject can be compared to obtain information regarding the prostate cancer state of the test subject.

Using the purified markers or their nucleic acid sequences, antibodies that specifically bind to a marker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). Such techniques include, but are not limited to, antibody preparation by selection of antibodies from libraries of recombinant antibodies in phage or similar vectors, as well as preparation of polyclonal and monoclonal antibodies by immunizing rabbits or mice (see, e.g. Huseetal., Science 246:1275-1281 (1989); Ward et al., Nature 341:544-546 (1989)). Typically a specific or selective reaction will be at least twice background signal or noise and more typically more than 10 to times background.

Generally, a sample obtained from a subject can be contacted with the antibody that specifically binds the marker. Optionally, the antibody can be fixed to a solid support to facilitate washing and subsequent isolation of the complex, prior to contacting the antibody with a sample. Examples of solid supports include glass or plastic in the fomm of, e.g. a microtiter plate, a stick, a bead, or a microbead.

Antibodies can also be attached to a probe substrate or biochip array. The sample is preferably a biological fluid sample taken from a subject. The sample can be diluted with a suitable eluant before contacting the sample to the antibody. After incubating the sample with antibodies, the mixture is washed and the antibody-marker complex formed can be detected. This can be accomplished by incubating the washed mixture with a detection reagent. This detection reagent may be, e.g. a second antibody which is labeled with a detectable label. Exemplary detectable labels include magnetic beads (e.g. DYNABEADS1M), fluorescent dyes, radiolabels, enzymes (e.g. horse radish peroxide, alkaline phosphatase and others commonly used in an ELISA), and calorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker is incubated simultaneously with the mixture.

Methods for measuring the amount of or presence of, antibody-marker complex include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non imaging methods. Electrochemical methods include voltametry and amperometry methods. Radio frequency methods include multipolar resonance spectroscopy. Methods for performing these assays are readily known in the art. Useful assays include, for example, an enzyme immune assay (EIA) such as enzyme-linked immunosorbent assay (ELISA), a radioimmune assay (RIA), a Western blot assay, or a slot blot assay. These methods are also described in, e.g. Methods in Cell Biology: Antibodies in Cell Biology, volume 37 (Asai, ed. 1993); Basic and Clinical Immunology (Stiles & Terr, eds., 7th ed. 1991); and Harlow & Lane, supra.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10C to 40° C. Immunoassays can be used to determine presence or absence of a marker in a sample as well as the quantity of a marker in a sample. The amount of an antibody marker complex can be determined by comparing to a standard. A standard can be, e.g. a known compound or another protein known to be present in a sample. The test amount of marker need not be measured in absolute units, as long as the unit of measurement can be compared to a control.

The methods for detecting these markers in a sample have many applications. For example, one or more markers can be measured to aid human cancer diagnosis or prognosis. In another example, the methods for detection of the markers can be used to monitor responses in a subject to cancer treatment. In another example, the methods for detecting markers can be used to assay for and to identify compounds that modulate expression of these markers in vivo or in vitro. In a preferred example, the biomarkers are used to differentiate between the different stages of tumor progression, thus aiding in determining appropriate treatment and extent of metastasis of the tumor.

Analysis of Biological Patterns

The output from a detection device can then be processed, stored, and further analyzed or assayed, e.g., using a bioinformatics system. A bioinformatics system can include one or more of the following: a computer; a plurality of computers connected to a network; a signal processing tool(s); and a pattern recognition tool(s). These tools can be present within the detection device or can be connected to the detection device or can be stand-alone tools into which a user inputs the information obtained from a detection device.

Signal processing utilizes mathematical foundations to align, scale, remove noise from, and reduce the dimensionality of the data. Signal processing may involve any of the following procedures, including alignment, scaling, transformation (e.g. log or square-root transforms), noise removal, and dimensionality reduction. Dynamic programming or regression methods can be used to align a separation axis with a standard separation profile. Intensities may be normalized, and/or scaled, to allow appropriate comparisons. The data sets can then be transformed using wavelets and/or other mathematical techniques that may be specifically designed for separation and mass spectrometer data to remove noise and leave informative signals. In a preferred embodiment, signal, processing filters out noise, leaving informative signals, and reduces spectrum dimensionality.

In some embodiments, signal processing may also involve the calibration of a mass-axis using linear correction determined by the calibrants. Calibration can take place prior to any sample detection; after sample detection; or in recurring intervals, for example.

Following signal processing, pattern recognition tools can be utilized to identify a pattern of subtle differences between phenotypic states. In some preferred embodiments, the pattern is used to make a decision regarding the prostate cancer state of a patient. “Prostate cancer state” is used herein to refer to the status of prostate cancer in the patient being studied. This state can include the absence or the presence of prostate cancer. Also, the various states include different forms of prostate cancer. Also, the prostate cancer state of a patient can be modified based on various treatment regimes being used on the patient. A pattern is obtained by training a pattern recognition algorithm on a sample of the data. The features that comprise the pattern discriminate the subtle differences between phenotypic states. In some embodiments, the data is sampled many times to obtain statistics on the patterns. These statistics and patterns are used to identify markers that constitute the biological pattern. In other embodiments, a metric is calculated, describing the discriminatory power of each point in the data, to identify markers that constitute the biological pattern.

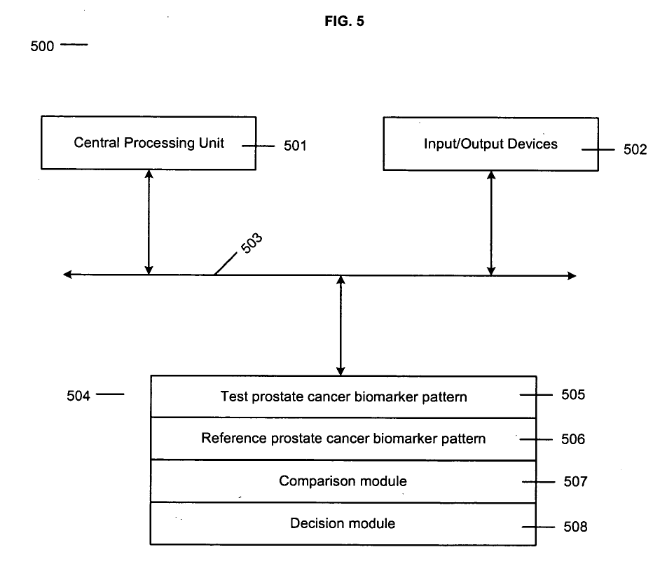

In some embodiments, the methods of the present invention are performed using a computer as depicted in FIG. 5. FIG. 5 illustrates a computer for implementing selected operations associated with the methods of the present invention. The computer 500 includes a central processing unit 501 connected to a set of input/output devices 502 via a system bus 503. The input/output devices 502 may include a keyboard, mouse, scanner, data port, video monitor, liquid crystal display, printer, and the like. A memory 504 in the form of primary and/or secondary memory is also connected to the system bus 503. These components of FIG. 5 characterize a standard computer. This standard computer is programmed in accordance with the invention. In particular, the computer 500 can be programmed to perform various operations of the methods of the present invention, for example, the processing operations of FIGS. 3, 4, and 7.

In some embodiments, the memory 504 of the computer 500 stores test 505 and reference 506 biomarker patterns. The memory 504 also stores a comparison module 507. The comparison module 507 includes a set of executable instructions that operate in connection with the central processing unit 501 to compare the various biomarker patterns. In other words, the comparison module 507 can perform the operation associated with step 304 of FIG. 3 or step 403 of FIG. 4 or step 705 of FIG. 7. The executable code of the comparison module 507 may utilize any number of numerical techniques to perform the comparisons.

The memory 504 also stores a decision module 508. The decision module 508 includes a set of executable instructions to process data created by the comparison module 507. The executable code of the decision module 508 may be incorporated into the executable code of the comparison module 507, but these modules are shown as being separate for the purpose of illustration. In preferred embodiments, the decision module 508 includes executable instructions to provide a decision regarding the prostate cancer state of a patient. Preferably, the decision module 508 performs operations associated with step 305 of FIG. 3 or step 404 of FIG. 4 or step 706 of FIG. 7.

In one embodiment, deciding whether a test sample comes from a patient that has prostate cancer is computed as follows: identify the intensity levels for biomarkers in Table 1 for the reference samples and for the test sample. The reference samples are those samples defined in the study design. Sum together the intensities for all charge states for a given biomarker. This yields a set of summed intensities, two intensities for every sample. Let the intensities for the test sample be identified by T=(biomarker 1 intensity for test sample, biomarker 2 intensity for test sample). Let the intensities for each of the reference samples be identified by R(i)=(biomarker 1 intensity for sample i, biomarker 2 intensity for sample i). A comparison between the test sample, T, and reference sample, R(i), is done by taking a dot product between the two: (T*R(i))=(biomarker 1 intensity for test sample)*(biomarker 1 intensity for sample i)+(biomarker 2 intensity for test sample)*(biomarker 2 intensity for sample i)

A decision function, D, is made from these comparisons by computing a function that appropriately weights them:

$$D = {\sum }^{} {α}_{i} ( T * R ( i ) ) + b$$

The αᵢ and b parameters are numbers that are appropriate for deciding whether the patient has prostate cancer based on the reference samples. The decision is made that the patient has prostate cancer if the function D is greater than 0 and that the patient does not, have prostate cancer if the function D is less than or equal to 0.

A computer readable medium with information regarding one or more of biomarkers 1-47 can be used in the diagnosis of prostate cancer. Preferably, the medium contains a reference pattern of one or more of biomarkers 1-47. This reference pattern is used to compare the pattern obtained from a test subject and a diagnosis of prostate cancer is made based on this comparison. This reference pattern can be from normal subjects, i.e., subjects with no prostate cancer, subjects with different levels of PSA, subjects with prostate cancer of varying severity. These reference patterns can be used for diagnosis, prognosis, evaluating efficacy of cancer treatment, and/or determining the severity of the prostate cancer condition of a subject. The methods of the present invention also include sending information regarding one or more of biomarkers 1-47 for use in the diagnosis of prostate cancer between one or more computers, for example with the use of the internet.

Markers and Patterns of Cancer Markers

In the present invention, markers and preferably patterns of biological markers, specifically prostate cancer markers, are analyzed. Also, novel prostate cancer marker patterns that have been identified are described herein.

In some embodiments, prostate cancer markers are identified in a biological sample from an animal subject and these markers are used to make a decision regarding the prostate cancer state of the subject. Typically, the animal subject is a human patient. Preferably, the markers used in the analysis are characterized by one or more mass spectral signals. Typically, the mass spectral signals are mass spectrum peaks obtained using a mass spectrometry system and are characterized by m/z values, molecular weights, and/or charge states, and/or migration times.

The prostate cancer markers—of the invention are characterized by the molecular weight and/or mass spectral data provided in the following tables. Table 1 lists the biomarkers and their corresponding molecular weights. Two preferred prostate cancer markers are provided in Table 2 along with their m/z values. One or both of the markers of Table—2 are preferably utilized in the present invention. The markers utilized are those that produce the approximate m/z values in Tables 2 or 3, assuming the experimental conditions disclosed in the Examples section are utilized;—however, any suitable detection methods other than mass spectroscopy may be utilized to detect these makers—characterized by the m/z values set forth in the tables.

TABLE 1

| | Molecular Weight |
|---|---|
| Biomarker | (Daltons) |
| 1* | 294 |
| 2 | 13880 |
| 3 | 1050 |
| 4 | 519 |
| 5 | 9061 |
| 6 | 4201 |
| 7* | 496 |
| 8 | 3331 |
| 9 | 2162 |
| 10 | 6169 |
| 11 | 3307 |
| 12 | 9288 |
| 13 | 7728 |
| 14 | 9289 |
| 15 | 3224 |
| 16 | 764 |
| 17* | 618 |
| 18 | 5720 |
| 19 | 1397 |
| 20 | 11439 |
| 21 | 14043 |
| 22 | 1626 |
| 23* | 333 |
| 24 | 13727 |
| 25 | 13876 |
| 26* | 228 |
| 27* | 326 |
| 28 | 965 |
| 29* | 256 |
| 30 | 624 |
| 31 | 894 |
| 32 | 856 |
| 33 | 12451 |
| 34 | 1855 |
| 35 | 11729 |
| 36 | 13897 |
| 37 | 13841 |
| 38 | 13978 |
| 39 | 6630 |
| 40* | 686 |
| 41* | 312 |
| 42 | 1465 |
| 43 | 981 |
| 44 | 943 |
| 45* | 272 |
| 46* | 228 |
| 47* | 341 |

*molecular weight for the indicated entities is as shown or +1 dalton

TABLE 2

| Biomarker | | | | | | |
|---|---|---|---|---|---|---|
| (*molecular weight for the | | | | | Separation | |
| indicated monoisotopic | | | | | Time (sec) | up or down |
| entities is as shown or +1 | | Observed m/z | monoisotopic* or | Molecular Weight | (+/−64 sec for | regulated in |
| dalton) | Charge | (thomson) | average for m/z | (Daltons) | 95% CI) | cancer cells |
| 1* | 1 | 2.9511E+02 | monoisotopic | 294 | 214 | down |
| 2 | 9 | 1.5433E+03 | average | 13880 | 452 | up |
| | 10 | 1.3890E+03 | average | 13880 | 452 | |
| | 11 | 1.2629E+03 | average | 13880 | 452 | |
| | 12 | 1.1577E+03 | average | 13880 | 452 | |
| | 13 | 1.0687E+03 | average | 13880 | 452 | |
| | 14 | 9.9246E+02 | average | 13880 | 452 | |
| | 15 | 9.2636E+02 | average | 13880 | 452 | |
| | 16 | 8.6852E+02 | average | 13880 | 452 | |
| | 17 | 8.1749E+02 | average | 13880 | 452 | |
| | 18 | 7.7213E+02 | average | 13880 | 452 | |
| | 19 | 7.3155E+02 | average | 13880 | 452 | |
| | 20 | 6.9502E+02 | average | 13880 | 452 | |
| | 21 | 6.6197E+02 | average | 13880 | 452 | |

Markers 3-47 are presented in Tables 3A-3D along with their m/z values.

TABLE 3A

| Biomarker | | | | | | |
|---|---|---|---|---|---|---|
| (*molecular weight for | | | | | Separation | |
| the indicated | | | | | Time (sec) | up or down |
| monoisotopic entities is | | Observed m/z | monoisotopic* or | Molecular Weight | (+/−64 sec for | regulated in |
| as shown or +1 dalton) | Charge | (thomson) | average for m/z | (Daltons) | 95% CI) | cancer cells |
| 3 | 2 | 5.2576E+02 | monoisotopic | 1050 | 230 | down |
| 4 | 1 | 5.2035E+02 | monoisotopic | 519 | 192 | down |
| | 2 | 2.6067E+02 | monoisotopic | 519 | 192 | |
| 5 | 8 | 1.1336E+03 | average | 9061 | 708 | up |
| | 9 | 1.0077E+03 | average | 9061 | 708 | |
| | 10 | 9.0707E+02 | average | 9061 | 708 | |
| 6 | 4 | 1.0513E+03 | monoisotopic | 4201 | 341 | up |
| | 5 | 8.4127E+02 | monoisotopic | 4201 | 341 | |
| 7* | 1 | 4.9723E+02 | monoisotopic | 496 | 279 | down |
| 8 | 3 | 1.1113E+03 | monoisotopic | 3331 | 452 | up |
| | 4 | 8.3369E+02 | monoisotopic | 3331 | 452 | |
| | 5 | 6.6715E+02 | monoisotopic | 3331 | 452 | |
| 9 | 3 | 7.2164E+02 | monoisotopic | 2162 | 495 | up |
| | 4 | 5.4148E+02 | monoisotopic | 2162 | 495 | |
| 10 | 6 | 1.0291E+03 | average | 6169 | 452 | up |
| | 7 | 8.8222E+02 | average | 6169 | 452 | |
| | 8 | 7.7207E+02 | average | 6169 | 452 | |
| 11 | 4 | 8.2773E+02 | monoisotopic | 3307 | 331 | up |
| 12 | 7 | 1.3279E+03 | average | 9288 | 643 | up |
| | 8 | 1.1620E+03 | average | 9288 | 643 | |
| | 9 | 1.0330E+03 | average | 9288 | 643 | |
| | 10 | 9.2982E+02 | average | 9288 | 643 | |
| 13 | 7 | 1.1050E+03 | average | 7728 | 400 | up |
| | 8 | 9.6701E+02 | average | 7728 | 400 | |
| | 9 | 8.5967E+02 | average | 7728 | 400 | |
| 14 | 7 | 1.3279E+03 | average | 9289 | 633 | up |
| | 8 | 1.1621E+03 | average | 9289 | 633 | |
| | 9 | 1.0331E+03 | average | 9289 | 633 | |
| | 10 | 9.2986E+02 | average | 9289 | 633 | |
| 15 | 4 | 8.0696E+02 | monoisotopic | 3224 | 564 | up |
| | 5 | 6.4576E+02 | monoisotopic | 3224 | 564 | |
| 16 | 1 | 7.6536E+02 | monoisotopic | 764 | 235 | down |
| | 2 | 3.8318E+02 | monoisotopic | 764 | 235 | |
| 17* | 1 | 6.1935E+02 | monoisotopic | 618 | 265 | up |
| 18 | 6 | 9.5430E+02 | average | 5720 | 483 | up |
| | 7 | 8.1812E+02 | average | 5720 | 483 | |
| | 8 | 7.1598E+02 | average | 5720 | 483 | |
| | 9 | 6.3653E+02 | average | 5720 | 483 | |

TABLE 3B

| Biomarker | | | | | | |
|---|---|---|---|---|---|---|
| (*molecular weight for the | | | | | Separation | |
| indicated monoisotopic | | | | | Time (sec) | up or down |
| entities is as shown or +1 | | Observed m/z | monoisotopic* or | Molecular Weight | (+/−64 sec for | regulated in |
| dalton) | Charge | (thomson) | average for m/z | (Daltons) | 95% CI) | cancer cells |
| 19 | 2 | 6.9929E+02 | monoisotopic | 1397 | 246 | up |
| 20 | 12 | 9.5422E+02 | average | 11439 | 482 | up |
| | 13 | 8.8089E+02 | average | 11439 | 482 | |
| | 14 | 8.1804E+02 | average | 11439 | 482 | |
| | 15 | 7.6357E+02 | average | 11439 | 482 | |
| | 16 | 7.1591E+02 | average | 11439 | 482 | |
| | 17 | 6.7386E+02 | average | 11439 | 482 | |
| | 18 | 6.3648E+02 | average | 11439 | 482 | |
| 21 | 13 | 1.0812E+03 | average | 14043 | 451 | up |
| | 14 | 1.0040E+03 | average | 14043 | 451 | |
| | 15 | 9.3718E+02 | average | 14043 | 451 | |
| | 16 | 8.7867E+02 | average | 14043 | 451 | |
| | 17 | 8.2704E+02 | average | 14043 | 451 | |
| | 18 | 7.8115E+02 | average | 14043 | 451 | |
| | 19 | 7.4009E+02 | average | 14043 | 451 | |
| 22 | 3 | 5.4295E+02 | monoisotopic | 1626 | 470 | up |
| | 4 | 4.0747E+02 | monoisotopic | 1626 | 470 | |
| 23* | 1 | 3.3413E+02 | monoisotopic | 333 | 296 | up |
| 24 | 13 | 1.0569E+03 | average | 13727 | 455 | up |
| | 14 | 9.8152E+02 | average | 13727 | 455 | |
| | 15 | 9.1615E+02 | average | 13727 | 455 | |
| | 16 | 8.5896E+02 | average | 13727 | 455 | |
| | 17 | 8.0849E+02 | average | 13727 | 455 | |
| | 18 | 7.6363E+02 | average | 13727 | 455 | |
| | 19 | 7.2349E+02 | average | 13727 | 455 | |
| 25 | 14 | 9.9214E+02 | average | 13876 | 494 | up |
| | 15 | 9.2607E+02 | average | 13876 | 494 | |
| | 16 | 8.6825E+02 | average | 13876 | 494 | |
| | 17 | 8.1723E+02 | average | 13876 | 494 | |
| | 18 | 7.7189E+02 | average | 13876 | 494 | |
| 26* | 1 | 2.2911E+02 | monoisotopic | 228 | 193 | down |
| 27* | 1 | 3.2712E+02 | monoisotopic | 326 | 194 | up |
| 28 | 2 | 4.8368E+02 | monoisotopic | 965 | 199 | up |
| 29* | 1 | 2.5715E+02 | monoisotopic | 256 | 199 | down |
| 30 | 1 | 6.2533E+02 | monoisotopic | 624 | 306 | up |
| | 2 | 3.1316E+02 | monoisotopic | 624 | 306 | |
| | 3 | 2.0911E+02 | monoisotopic | 624 | 306 | |
| 31 | 2 | 4.4813E+02 | monoisotopic | 894 | 235 | down |

TABLE 3C

| Biomarker | | | | | | |
|---|---|---|---|---|---|---|
| (*molecular weight for the | | | | | Separation | |
| indicated monoisotopic | | | | | Time (sec) | up or down |
| entities is as shown or +1 | | Observed m/z | monoisotopic* or | Molecular Weight | (+/−64 sec for | regulated in |
| dalton) | Charge | (thomson) | average for m/z | (Daltons) | 95% CI) | cancer cells |
| 32 | 1 | 8.5739E+02 | monoisotopic | 856 | 235 | down |
| | 2 | 4.2920E+02 | monoisotopic | 856 | 235 | |
| 33 | 7 | 1.7797E+03 | average | 12451 | 373 | up |
| | 8 | 1.5574E+03 | average | 12451 | 373 | |
| | 9 | 1.3845E+03 | average | 12451 | 373 | |
| 34 | 3 | 6.1932E+02 | monoisotopic | 1855 | 328 | up |
| 35 | 10 | 1.1739E+03 | average | 11729 | 601 | up |
| | 11 | 1.0673E+03 | average | 11729 | 601 | |
| | 12 | 9.7840E+02 | average | 11729 | 601 | |
| | 13 | 9.0322E+02 | average | 11729 | 601 | |
| | 14 | 8.3878E+02 | average | 11729 | 601 | |
| 36 | 13 | 1.0700E+03 | average | 13897 | 451 | up |
| | 14 | 9.9366E+02 | average | 13897 | 451 | |
| | 15 | 9.2748E+02 | average | 13897 | 451 | |
| | 16 | 8.6957E+02 | average | 13897 | 451 | |
| | 17 | 8.1848E+02 | average | 13897 | 451 | |
| | 18 | 7.7307E+02 | average | 13897 | 451 | |
| | 19 | 7.3243E+02 | average | 13897 | 451 | |
| | 20 | 6.9586E+02 | average | 13897 | 451 | |
| 37 | 11 | 1.2593E+03 | average | 13841 | 443 | up |
| | 12 | 1.1544E+03 | average | 13841 | 443 | |
| | 13 | 1.0657E+03 | average | 13841 | 443 | |
| | 14 | 9.8967E+02 | average | 13841 | 443 | |
| | 15 | 9.2376E+02 | average | 13841 | 443 | |
| | 16 | 8.6609E+02 | average | 13841 | 443 | |
| | 17 | 8.1520E+02 | average | 13841 | 443 | |
| | 18 | 7.6997E+02 | average | 13841 | 443 | |
| | 19 | 7.2949E+02 | average | 13841 | 443 | |

TABLE 3D

| Biomarker | | | | | Separation | |
|---|---|---|---|---|---|---|
| (*molecular weight for the | | | | | Time (sec) | up or down |
| indicated monoisotopic entities | | Observed m/z | monoisotopic* or | Molecular Weight | (+/−64 sec for | regulated in |
| is as shown or +1 dalton) | Charge | (thomson) | average for m/z | (Daltons) | 95% CI) | cancer cells |
| 38 | 11 | 1.2717E+03 | average | 13978 | 452 | up |
| | 12 | 1.1659E+03 | average | 13978 | 452 | |
| | 13 | 1.0762E+03 | average | 13978 | 452 | |
| | 14 | 9.9944E+02 | average | 13978 | 452 | |
| | 15 | 9.3288E+02 | average | 13978 | 452 | |
| | 16 | 8.7464E+02 | average | 13978 | 452 | |
| | 17 | 8.2325E+02 | average | 13978 | 452 | |
| | 18 | 7.7757E+02 | average | 13978 | 452 | |
| 39 | 6 | 1.1060E+03 | average | 6630 | 585 | up |
| | 7 | 9.4818E+02 | average | 6630 | 585 | |
| | 8 | 8.2978E+02 | average | 6630 | 585 | |
| | 9 | 7.3769E+02 | average | 6630 | 585 | |
| | 10 | 6.6402E+02 | average | 6630 | 585 | |
| | 11 | 6.0375E+02 | average | 6630 | 585 | |
| 40* | 1 | 6.8650E+02 | monoisotopic | 686 | 195 | up |
| 41* | 1 | 3.1314E+02 | monoisotopic | 312 | 305 | up |
| 42 | 2 | 7.3335E+02 | monoisotopic | 1465 | 266 | down |
| | 3 | 4.8924E+02 | monoisotopic | 1465 | 266 | |
| | 4 | 3.6718E+02 | monoisotopic | 1465 | 266 | |
| 43 | 2 | 4.9167E+02 | monoisotopic | 981 | 198 | up |
| 44 | 1 | 9.4442E+02 | monoisotopic | 943 | 198 | up |
| | 2 | 4.7271E+02 | monoisotopic | 943 | 198 | |
| 45* | 1 | 2.7310E+02 | monoisotopic | 272 | 192 | down |
| 46* | 1 | 229.1146625 | monoisotopic | 228 | 337 | down |
| 47* | 1 | 342.145859 | monoisotopic | 341 | 440 | up |

The molecular weights of the biomarkers are as indicated in the Tables herein or are about ±1 Dalton. Further, the m/z values are as indicated or the closest nominal mass.

The m/z values provided in the above Tables 2 and 3 are peaks that are obtained for the markers using mass spectrometry system under the conditions disclosed in the Examples section. Tables 2 and 3 indicate whether the levels of the markers were up or down in prostate cancer. It is intended herein that the methods of the invention are not limited to the up or down levels indicated in the Tables. The invention encompasses the determination of the differential presence of one or more biomarkers 1-47 for the diagnosis of prostate cancer. The differences in the levels of biomarkers are typically obtained by comparison to samples from normal subjects. The presence, absence, and/or levels of biomarkers 1-47 can be used in the diagnosis of prostate cancer. In preferred embodiments, one or more of the biomarkers 1-47 are used to make a decision regarding a prostate cancer state in a subject with serum PSA levels of less than about 6 ng/ml.

A marker may be represented at multiple m/z points in a spectrum. This can be due to the fact that multiple isotopes of the marker are observed and/or that multiple charge states of the marker are observed, or that multiple isoforms of the marker are observed. An example of different isoforms of the same marker is a protein that exists with and without a post-translational modification such as glycoslyation. These multiple representation of a marker can be analyzed individually or grouped together. An example of how multiple representations of a marker may be grouped is that the intensities for the multiple peaks can be summed.

It is intended herein that the methods of the present invention include identification of the markers of Table 1 and also any suitable different forms of the markers. For example, proteins are known to exist in a sample in a plurality of different forms characterized by different mass. These forms can result from either, or both, of pre- and post-translational modification. Pre-translational modified forms include allelic variants, slice variants and RNA editing forms. Post translationally modified forms include forms resulting from proteolytic cleavage (e.g., fragments of a parent protein), glycosylation, phosphorylation, lipidation, oxidation, methylation, cystinylation, sulphonation and acetylation. Thus, the invention includes the use of modified forms of the markers of Table 1 to diagnose prostate cancer.

The markers that are characterized by the mass spectral data provided in Tables 2 and 3 above can be identified using different techniques that are known in the art. These techniques are not limited to mass spectrometry systems and include immunoassays, protein chips, multiplexed immunoassays, and complex detection with aptamers and chromatography utilizing spectrophotometric detection.

The markers of Table 1 can be further characterized using techniques known in the art. For example, polypeptide markers can be further characterized by sequencing them using enzymes or mass spectrometry techniques. For example, see, Stark, in: Methods in Enzymology, 25:103-120 (1972); Niall, in: Methods in Enzymology, 27:942-1011 (1973); Gray, in: Methods in Enzymology, 25:121-137 (1972); Schroeder, in: Methods in Enzymology, 25:138-143 (1972); Creighton, Proteins: Structures and Molecular Principles (W. H. Freeman, NY, 1984); Niederwieser, in: Methods in Enzymology, 25:60-99 (1972); and Thiede, et al. FEBS Lett., 357:65-69 (1995), Shevchenko, A., et al., Proc. Natl. Acad. Sci. (USA), 93:14440-14445 (1996); Wilm, et al., Nature, 379:466-469 (1996); Mark, J., “Protein structure and identification with MS/MS,” paper presented at the PE/Sciex Seminar Series, Protein Characterization and Proteomics: Automated high throughput technologies for drug discovery, Foster City, Calif. (March, 1998); and Bieman, Methods in Enzymology, 193:455-479 (1990).

The markers of Table 1 are preferably used to diagnose prostate cancer in subjects with PSA levels of about 1 ng/ml to about 6 ng/ml. Most preferably, the markers described herein are used in the diagnosis of subjects with PSA levels of about 1 ng/ml to about 4 ng/ml. In some embodiments, the markers described herein are used in combination with the use of PSA as a marker. In one method, following a PSA test and a detection of PSA levels of about 6 ng/ml or lower, prior to a trans-rectal ultrasound and/or a biopsy, one or more markers of Table 1 are evaluated. Preferably in this method, following determination of PSA levels, transthyretin levels are analyzed.

In one embodiment, the markers described herein are identified in serum samples which are analyzed, separated, and introduced into a mass spectrometer. Preparation can include removal of high abundance proteins, addition of preservatives and calibrants, and desalting. Prepared samples are then separated using microfluidic based capillary electrophoresis (CE). Using an electrospray ionization (ESI) interface, samples are ionized and sprayed into a time of flight mass spectrometer. The resulting data for each sample is a series of mass spectra, acquired during the electrophoretic separation.

In some embodiments, the prostate cancer markers used to make a decision associated with the prostate cancer state of a patient involves the identification of a set of markers. The set can include one or more markers.

Typically, when patterns of prostate cancer markers are used to determine the prostate cancer state, the pattern from a patient, also referred to as test pattern, is compared mathematically to a set of reference patterns. The reference patterns can be derived from the same patient, different patient, or group of patients. In some embodiments, the reference patterns are obtained from normal subjects, i.e. subjects who do not have prostate cancer, as well as from subjects having prostate cancer.

A decision associated with the prostate cancer state of a patient can be made by analyzing a biological sample from a patient for patterns of prostate cancer markers using a mass spectrometry system. In one embodiment, the analysis of the samples does not involve separation on a protein affinity chip and preferably the markers are proteins, protein fragments, peptides, or small molecules. In some preferred embodiments, the samples are prepared and/or separated on a micro-fluidic device and/or delivered to the mass spectrometer by electrospray ionization.

The patterns from a subject suspected of having prostate cancer, in some embodiments, can be compared to reference patterns, which are typically obtained from one or more normal subjects. Also, patterns from the same patient can be compared to each other. Typically, these patterns are obtained at different time points and are used to evaluate the status of prostate cancer in the patient.

In some embodiments, subsets of prostate cancer markers identified herein are used in the classification of prostate cancer states. These subsets can comprise one or more markers described herein. Preferably the subset comprises one marker, preferably about 2 to about 10 markers, more preferable about 10 to about 50 markers, and even more preferably about 50 to about 150 markers.

In other embodiments, the markers described herein are used in combination with known prostate cancer markers. Several prostate cancer markers are known in the art. For example, see Tumor Markers, Physiology, Pathobiology, Technology and Clinical Applications, Editors E. P. Diamandis et al., AACC Press, vol. 36(4), 2003. Examples of known prostate cancer markers that can be used in combination with the markers described herein include, but are not limited to, prostate specific antigen (PSA), human glandular kallikrein 2, acid phosphatase (PAP, ACPP, ACP3), prostate-specific membrane antigen, androgen receptor, and insulin-like growth factors and binding proteins.

In yet other embodiments, the methods described herein are used in combination with known diagnostic techniques for prostate cancer. Examples of other diagnostic techniques include, but are not limited to, digital rectal exam (DRE), prostate biopsy, transrectal ultrasound (TRUS), computed tomography (CT) scan, and magnetic resonance imaging (MRI) scan.

Uses of Markers

In addition to being used for clinical purposes, the markers and patterns of markers have many other applications. The markers identified herein may be entire proteins or fragments of proteins or other analytes. It is intended herein that a particular marker not only encompass the protein fragment, but also the entire parent protein.

The markers and their patterns described herein can be used in the prognosis and treatment of prostate cancer and also in assays to identify and develop novel therapies for prostate cancer. In some embodiments, the biomarkers are used in assays to develop prostate cancer treatments. These treatments include, but are not limited to, antibodies, nucleic acid molcules (e.g., DNA, RNA, RNA antisense), peptides, peptidomimetics, and small molecules.

The markers found in the invention can be used to enable or assist in the pharmaceutical drug development process for therapeutic agents for use in prostate cancer. The markers can be used to diagnose disease for patients enrolling in a clinical trial. The markers can indicate the prostate cancer state of patients undergoing treatment in clinical trials, and show changes in the prostate cancer state during the treatment. The markers can demonstrate the efficacy of a treatment, and be used as surrogate endpoints for clinical trial outcome. The markers can be used to stratify patients according to their responses to various therapies.

One embodiment includes antibodies that bind to, and thereby affect the function of, these biomarkers. In other embodiments, cellular expression of the target marker can be modulated, for example, by affecting transcription and/or translation. Suitable agents include anti-sense constructs prepared using antisense technology or gene transcription constructs, such as using RNA interference technology. Also, DNA oligonucleotides can be designed to be complementary to a region of the gene involved in transcription thereby preventing transcription and the production of one or more of the biomarkers. Therapeutic and/or prophylactic polynucleotide molecules can be delivered using gene transfer and gene therapy technologies.

Still other agents include small molecules that bind to or interact with the biomarkers and thereby affect the function thereof, such as an agonist, partial agonist, or antagonist, and small molecules that bind to or interact with nucleic acid sequences encoding the biomarkers, and thereby affect the expression of these protein biomarkers. These agents may be administered alone or in combination with other types of treatments known and available to those skilled in the art for treating prostate cancer (e.g., radiation therapy, chemotherapy, hormonal therapy, immunotherapy and anti-tumor agents).

One aspect of the invention is therapeutic agents for use in prostate cancer patients. The therapeutic agents can be used either therapeutically, prophylactically, or both. Preferably, the therapeutic agents have a beneficial effect on the prostate cancer state of a patient. Even more preferably, the markers in Table 1 are used as targets for therapeutic agents. For markers that are polypeptides, the therapeutic agents may target the polypeptide or the DNA and/or RNA encoding the polypeptide. The therapeutic agent either directly acts on the markers or modulates other cellular constituents which then have an effect on the markers. In some embodiments, the therapeutic agents either activate or inhibit the activity of the markers. In other embodiments, a marker listed in Table 1 or an antibody to a marker listed in Table 1 is used as the therapeutic or prophylactic agent. In these embodiments, the markers or antibodies used as the active agent may be modified to improve certain physical properties in order to improve their therapeutic or prophylactic activities. For example, the marker maybe chemically modified to improve bioavailability or its pharmacokinetic properties.

The prostate cancer therapeutic agents of the present invention can be co-administered with other active pharmaceutical agents that are used for the therapeutic and/or prophylactic treatment of prostate cancer. This co-administration can include simultaneous administration of the two agents in the same dosage form, simultaneous administration in separate dosage forms, and separate administration. For example, the prostate cancer therapeutic agents can be co-administered with chemotherapeutic agents that are used to treat cancer. These two agents can be formulated together in the same dosage form and administered simultaneously. Alternatively, they can be simultaneously administered or separately administered, wherein both the agents are present in separate formulations. In the separate administration protocol, the two agents may be administered a few minutes apart, or a few hours apart, or a few days apart.

The prostate cancer therapeutic agents of the present invention can be used in combination with the other prostate cancer therapies. Examples of prostate cancer therapies include, but are not limited to, surgery, radiation therapy, hormone therapy, and chemotherapy.

The term “treating” as used herein includes having a beneficial effect, i.e., achieving a therapeutic benefit and/or a prophylactic benefit. By therapeutic benefit is meant eradication, amelioration, or prevention of the underlying disorder being treated. For example, in a cancer patient, therapeutic benefit includes eradication or amelioration of the underlying cancer. Also, a therapeutic benefit is achieved with the eradication, amelioration, or prevention of one or more of the physiological symptoms associated with the underlying disorder such that an improvement is observed in the patient, notwithstanding that the patient may still be afflicted with the underlying disorder. For example, administration of prostate cancer therapeutic agents to a patient suffering from prostate cancer provides therapeutic benefit not only when the patient's prostate cancer marker count is decreased, but also when an improvement is observed in the patient with respect to other disorders that accompany prostate cancer like pain and incontinence. For prophylactic benefit, the therapeutic agents may be administered to a patient at risk of developing prostate cancer or to a patient reporting one or more of the physiological symptoms of prostate cancer, even though a diagnosis of prostate cancer may not have been made.

The therapeutic agents of the present invention are administered in an effective amount, i.e., in an amount effective to achieve therapeutic or prophylactic benefit. The actual amount effective for a particular application will depend on the patient (e.g., age, weight, etc.), the condition being treated, and the route of administration. Determination of an effective amount is well within the capabilities of those skilled in the art. The effective amount for use in humans can be determined from animal models. For example, a dose for humans can be formulated to achieve circulating and/or gastrointestinal concentrations that have been found to be effective in animals.

Preferably, the agents used for therapeutic and/or prophylactic benefit can be administered per se or in the form of a pharmaceutical composition. The pharmaceutical compositions comprise the therapeutic agents, one or more pharmaceutically acceptable carriers, diluents or excipients, and optionally additional therapeutic agents. The compositions can be formulated for sustained or delayed release. The compositions can be administered by injection, topically, orally, transdermally, rectally, or via inhalation. Preferably, the therapeutic agent or the pharmaceutical composition comprising the therapeutic agent is administered orally. The oral form in which the therapeutic agent is administered can include powder, tablet, capsule, solution, or emulsion. The effective amount can be administered in a single dose or in a series of doses separated by appropriate time intervals, such as hours.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Suitable techniques for preparing pharmaceutical compositions of the therapeutic agents of the present invention are well known in the art.

Therapeutic and Diagnostic Uses of Marker or Patterns of Cancer Markers

The complement of proteins, protein fragments, peptides, or other analytes present at any specific moment in time defines who and what an individual organism is at that moment, as well as the state of health or disease: the biological state. The biological state of a cancer patient reflects not only the presence and nature of the cancer, but the more general state of health and response of the affected individual to the disease.

The methods described herein can be used to identify the state of prostate cancer in a patient, i.e., the prostate cancer state. In one embodiment, the methods are used to detect the earliest stages of disease (e.g. stage I cancer detection). In other embodiments, the methods are used to grade the identified cancer. In one embodiment, the methods are used to diagnose the presence or absence of prostate cancer. The methods can be used to categorize the cancer based on the probability that the cancer will metastasize. Also, these methods can be used to predict the possibility of the cancer going into remission in a particular patient.

In certain embodiments, patients, health care providers, such as doctors and nurses, or health care managers, use the patterns of prostate cancer markers to make a diagnosis, prognosis, and/or select treatment options.

In other embodiments, the methods described herein can be used to predict the likelihood of response for any individual to a particular treatment, select a treatment, or to preempt the possible adverse effects of treatments on a particular individual (e.g. monitoring toxicology due to chemotherapy). Also, the methods can be used to evaluate the efficacy of treatments over time. For example, biological samples can be obtained from a patient over a period of time as the patient is undergoing treatment. The patterns from the different samples can be compared to each other to determine the efficacy of the treatment. Also, the methods described herein can be used to compare the efficacies of different prostate cancer therapies and/or responses to one or more treatments in different populations (e.g., different age groups, ethnicities, family histories, etc.).

In a preferred embodiment, a mass spectrometry system is used to analyze one or more markers of Table 1 to evaluate the prostate cancer state of a patient. Intensities for one or more of the markers are obtained from the mass spectrometry system and these intensities are used to make the decision regarding the prostate cancer state. The intensity for a particular maker is normalized and weighted based on the intensity values obtained in samples from previous normal and prostate cancer patients. The normalized and weighted intensities are summed for all the markers being studied and the resulting value is used to make the decision regarding the prostate cancer state. A value greater than zero can indicate, for example, that the patient is healthy and a value less than zero indicates the presence of prostate cancer. In general, the magnitude of the value is related to the severity grading of the prostate cancer state of the subject.

Kits for the Diagnosis of Prostate Cancer

In yet another aspect, the invention provides kits for diagnosis of prostate cancer, wherein the kits can be used to detect the markers of the present invention. For example, the kits can be used to detect any one or more of the markers described herein, which markers are differentially present in samples of a human cancer patient and normal subjects.

In one embodiment, a kit comprises a substrate comprising an adsorbent thereon, wherein the adsorbent is suitable for binding a marker, and instructions to detect the marker or markers by contacting a sample with the adsorbent and detecting the marker or markers retained by the adsorbent. In another embodiment, a kit comprises (a) an antibody that specifically binds to a marker; and (b) a detection reagent. In some embodiments, the kit may further comprise instructions for suitable operation parameters in the form of a label or a separate insert. Optionally, the kit may further comprise a standard or control information so that the test sample can be compared with the control information standard to determine if the test amount of a marker detected in a sample is a diagnostic amount consistent with a diagnosis of prostate cancer.

Use of Transthyretin as a Cancer Marker

In one embodiment, transthyretin is used as a prostate cancer marker in subjects with low PSA levels, for example, in subjects with PSA levels lower than about 6 ng/ml. In this diagnostic method, any suitable form of transthyretin can be used as a marker. In embodiments wherein a fragment of transthyretin is detected, the fragment is a suitable fragment such that it is characterized by one or more biological activities of transthyretin. In a preferred embodiment, the form of transthyretin with a mass of 13,880, amino acids 21-147, with a disulfide link to cysteine at Cys-30 is used as a biomarker for prostate cancer. In these embodiments, the thransthyretin can be used as a marker by itself or can be used as a marker in combination with known prostate cancer markers such as PSA or with one or more other markers from Table 1.

Transthyretin (also known as prealbumin; SwissProt entry TTHY_HUMAN; accession PO2766) is produced in the liver. It is a member of a class of proteins known as Acute Phase Proteins or Host Response Proteins. These proteins are part of a general immune response, and they change in response to trauma or infection. Transthyretin is a negative acute phase response protein, i.e., its abundance generally decreases as part of the acute phase response. Transthyretin is a carrier for serum thyroxine and triiodothyronine (thyroid hormones) and it facilitates the transport of retinol via its interaction with retinol binding proteins.

The transthyretin protein is expressed as a protein of 147 amino acids. The signal sequence is cleaved to form the mature protein of length 21-147. There is a free cysteine that has the potential to be modified. There are several known isoforms of the protein (see Fung et al, Int. J. Cancer, 2005): Unmodifed 21-147 (MW 13761); Cysteinylated 21-147 (MW 13880); Glutathionylated 21-147 (MW 14066) and Truncated 31-147 (MW 12843).

The protein normally exists as a homotetramer and this structure is necessary for its binding properties, and to avoid glomerular filtration. The Cys modification may interfere with tetramer formation. The Cys modification may be a result of altered redox chemistry in the cell.

Transthyretin can be detected in samples using techniques known in the art, including the mass spectrometry technique described herein. A sample to be analyzed is collected. Prior to analysis for tranthyretin, the sample is separated and/or prepared using known techniques, including those described herein. Transthyretin can also be detected using immunoassays that employ commercially available transthyretin antisera. Techniques for detection of transthyretin are described in Zhang et al., Cancer Res. 2004 Aug. 15;64(16):5882-90; Robey et al., J Urol. 1985 October;134(4):787-90.; Kajita et al., Endocrinol Jpn. 1981 December;28(6):785-91; Rostenberg et al., J Natl Cancer Inst. 1979 February;62(2):299-300; and Ward et al., Br J Urol. 1977 October;49(5):411-8.

The following example is intended to illustrate details of the invention, without thereby limiting it in any manner.

### EXAMPLE

Microfluidic-based capillary electrophoresis-mass spectrometry was used to identify prostate cancer markers. The objective was to find patterns which differentiate those individuals with prostate cancer from those without in subjects with a PSA value between 1-6 ng/ml.

Study Design

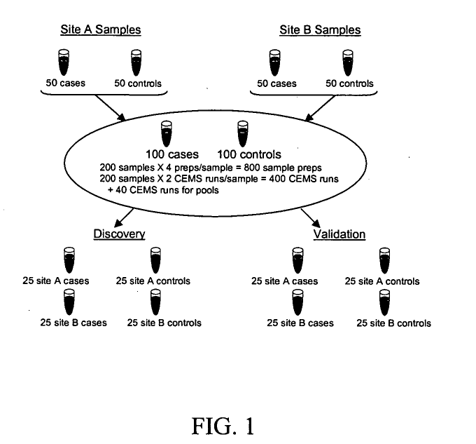

Samples were divided into discovery and validation sets. Data was collected from both sample sets concurrently. Data from the discovery samples was used to find a biomarker pattern, and data from the validation samples was used to evaluate how well the pattern can distinguish between the two groups of men (i.e. the validation data set was not used for training or testing in discovery cross-validation). Data was analyzed from each site's samples independently and then evaluated for overlap between the results. Table 4 provides a description of the samples and FIG. 1 provides a schematic overview of the samples.

Half of the 200 samples shown in FIG. 1 were used for Discovery of patterns, as described above. These included 25 case and 25 control samples from site A and 25 case and 25 control samples from site B. Following pattern discovery, the second half of the 200 samples shown in FIG. 1 was used for validation of the patterns. Validation consisted of determining whether, for each sample, a pattern correctly identifies the sample as prostate cancer (case) or non-prostate cancer (control), using the decision function, D, described above.

TABLE 4

Sites

| Sample | Site A | Site B |
|---|---|---|
| Disease Cases | 50 | 50 |
| Control Cases | 50 | 50 |

Sample Analysis

Serum samples were prepared, separated, and introduced into a mass spectrometer for analysis. Preparation included the removal of high abundance proteins, addition of preservatives and calibrants, and desalting. Prepared samples were then separated using microfluidic based capillary electrophoresis (CE) in a ˜12 minute separation. Using an electrospray ionization (ESI) interface, samples were ionized and sprayed directly into a time-of-flight mass spectrometer (MS). The resulting CE-MS data for each sample was a series of mass spectra, acquired during the electrophoretic separation. Samples were prepared and analyzed in a randomized order to minimize biases.

Sample Criteria

Samples were collected pre-biopsy and pre-treatment, and samples were collected either before or after DRE. If a DRE had been performed, samples were collected at least 24 hours post-DRE.

Matching of cases and controls was done based on site, PSA levels, age at sample collection, date of sample draw, and race, in that order of priority.

A volume of approx. 10 cc of venous blood was drawn in serum tubes (“red or marble” top glass tube, BD Vacutainer. After sitting for minimum of 30 minutes to a maximum of 12 hrs the sample was centrifuged and the serum was collected and frozen (−80° C.).

Approximately 200 μL of serum was required for analysis from each patient.

TABLE 5
Inclusion and Exclusion Criteria

| | Cases | |
|---|---|---|
| Objective | Inclusion | Exclusion |
| 1 | PSA values in the 1-6 ng/ml | Prior to entering this study |
| | range who have a confirmed | history of any other cancer, |
| | diagnosis of prostate cancer. | other than non-melanoma |
| | Reasons for biopsy of these | skin cancer. |
| | individuals may include rising | <40 years old |
| | PSA, abnormal DRE, or | Samples that have |
| | high-risk status (e.g., family | undergone more than 1 |
| | history of prostate cancer). | freeze/thaw cycle. |

Prostate cancer diagnosis was based on pathological analysis of at least one 6-core TRUS guided biopsy.

To be considered a control, patients had at least one 6-core TRUS guided biopsy that did not find evidence of prostate cancer.

Control Samples

Spiked serum A was a control run at the beginning of each day. This consisted of serum that had been processed following the standard sample prep protocol and spiked with components at specific concentrations post processing. Composition can be found in Table 6.

TABLE 6
Spiked Serum A components

Concentration (nM)

| | Effective | |
|---|---|---|
| | concentration in | Actual concentration |
| Standard | unprocessed serum | in resuspended serum |
| Pre-Processing | 100 | 1000 |
| Ala-met | | |
| enkephalin | | |
| Post-Processing | | |
| LHRH fragment | 300 | 3000 |
| Bradykinin | 300 | 3000 |
| Angiotensin III | 300 | 3000 |
| Ubiquitin | 300 | 3000 |
| Aprotinin | 300 | 3000 |
| Renin | 300 | 3000 |
| Neurotensin | 50 | 500 |

Sample Preparation and Data Collection

Each sample was prepared 4 times and run 2 times on the CE-MS.

The 200 samples were prepared four times each. The 4 replicates of each prepared sample were pooled and re-divided into 4 aliquots. Two of those aliquots were used in CE-MS.

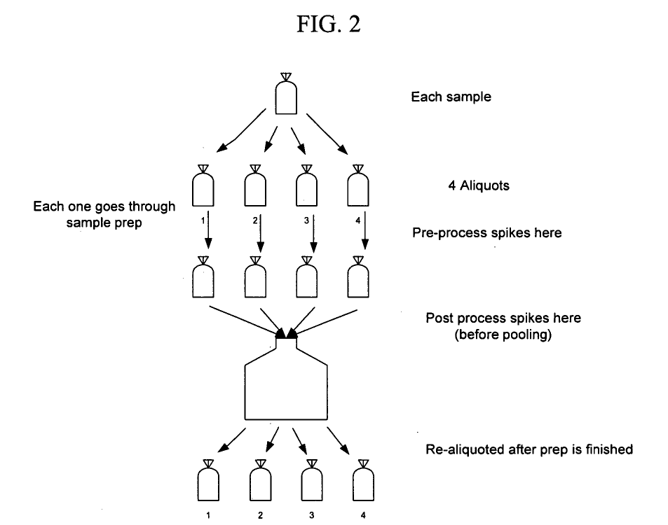

The standard sample preparation is outlined in FIG. 2. The composition of Sample Standard was 0.30 μM angiotensin III and 10.0 μM Aprotinin and Sample Diluent was 390 μL HPLC water, 50 μL 10% formic, 5 μL Pepstatin 1:10 in H₂O, 5 μL Sample Standard.

Samples were thawed sample for the run at room temperature and transfered to ice at once when thawed. Runs were set up in duplicate on each of two μElute plate (n=4 each sample). All samples were run individually. 450 μL of sample diluent was added to 50 μL of serum sample and mix. Diluted samples were transfered immediately to YM50 Microcon (within ten minutes) and centrifuged at 13,000×g for 30 minutes in the centrifuge with 45° angle black anodized rotor. 25 μL 10% trifluoroacetic acid was added just before application to reverse phase. Samples were processed on μElute plate and collected in PCR plate. Samples were dried in the vacuum centrifuge. Aliquots were re-suspended with 5 μL of re-suspension buffer of IPA and formic containing post-processing standard, bradykinin and renin at 3000 nM actual concentration in resuspended serum. Samples were vortexed for two minutes and centrifuged for 10 sec. After sample preparation the 4 separate preparations were pooled and re-aliquoted.

The mass spectrometer was set up with the inlet capillary voltage to 280, PMT bias to −770, and MCP bias to −6000 in the volts window. The scan range was set to 122496, Number of Scans to 8000, Acq. Bin Width to 1 and threshold to 35. The spiked serum sample was run in the CE-MS to verify the intensities, resolution and migration times for the standards.

The mass spectrometer was rinsed with sample and then loaded with a chip of 1 μM set 6 in 20% IPA, 0.05% formic acid for chip infusion. A single use vial is run of set 6 1 μM in 20% EPA 0.05% forming acid for chip infusion. After the pre-run is complete, the signal and resolution of the 1 μM neurotensin³⁺ peak at 558.3 m/z is monitored. The inlet lens voltage is adjusted in 0.05 V increments to obtain the optimum counts and resolution for neurotensin³⁺ (signal intensity: ≧150,000 counts; resolution: 6000-8000). When the intensity and resolution fall within these limits, another Spiked Serum A was run.

Sample runs: Samples are removed from −20° C. freezer and stored on ice during CE-MS runs for no longer than 4 hours. One sample is used to complete 1 CE-MS run and obtain the data. During sample runs, sprays were visually inspected for stability.

Data Analysis

CE-MS data were analyzed several ways after data quality assurance. Peaks were identified using several methods, including mass-spectrometry-specific signal processing methods. First, univariate statistics were used to find single peak and/or component intensities that correlate with the presence/absence of prostate cancer. Standard non-parametric methods were used due to small sample size and the inability to assume normality of data. Such methods include the Mann-Whitney test. Second, after ranking by P-value, results were visualized, and those peaks/components that have high group-mean differences were determined. Third, a suite of feature selection and pattern classification methods were used to find multi-variate patterns that distinguish between the presence and absence of prostate cancer. These methods include support vector machines, discriminant analysis, and other machine learning methods. Cross-validation techniques were utilized to train and test patterns. The sensitivities, specificities and positive/negative predictive values of patterns that can highly discriminate between classes were determined. Proteomic data were analyzed with and without PSA scores and other clinical measurements available.

The markers identified are shown in Tables 7 and 8 below.

TABLE 7

| Biomarker | | | | | | |
|---|---|---|---|---|---|---|
| (*molecular weight for | | | | | Separation | |
| the indicated | | | | | Time (sec) | up or down |
| monoisotopic entities is | | Observed m/z | monoisotopic* or | Molecular Weight | (+/−64 sec for | regulated in |
| as shown or +1 dalton) | Charge | (thomson) | average for m/z | (Daltons) | 95% CI) | cancer cells |
| 1* | 1 | 2.9511E+02 | monoisotopic | 294 | 214 | down |
| 2 | 9 | 1.5433E+03 | average | 13880 | 452 | up |
| | 10 | 1.3890E+03 | average | 13880 | 452 | |
| | 11 | 1.2629E+03 | average | 13880 | 452 | |
| | 12 | 1.1577E+03 | average | 13880 | 452 | |
| | 13 | 1.0687E+03 | average | 13880 | 452 | |
| | 14 | 9.9246E+02 | average | 13880 | 452 | |
| | 15 | 9.2636E+02 | average | 13880 | 452 | |
| | 16 | 8.6852E+02 | average | 13880 | 452 | |
| | 17 | 8.1749E+02 | average | 13880 | 452 | |
| | 18 | 7.7213E+02 | average | 13880 | 452 | |
| | 19 | 7.3155E+02 | average | 13880 | 452 | |
| | 20 | 6.9502E+02 | average | 13880 | 452 | |
| | 21 | 6.6197E+02 | average | 13880 | 452 | |

TABLE 8A

| Biomarker | | | | | | |
|---|---|---|---|---|---|---|
| (*molecular weight for | | | | | Separation | |
| the indicated | | | | | Time (sec) | up or down |
| monoisotopic entities is | | Observed m/z | monoisotopic* or | Molecular Weight | (+/−64 sec for | regulated in |
| as shown or +1 dalton) | Charge | (thomson) | average for m/z | (Daltons) | 95% CI) | cancer cells |
| 3 | 2 | 5.2576E+02 | monoisotopic | 1050 | 230 | down |
| 4 | 1 | 5.2035E+02 | monoisotopic | 519 | 192 | down |
| | 2 | 2.6067E+02 | monoisotopic | 519 | 192 | |
| 5 | 8 | 1.1336E+03 | average | 9061 | 708 | up |
| | 9 | 1.0077E+03 | average | 9061 | 708 | |
| | 10 | 9.0707E+02 | average | 9061 | 708 | |
| 6 | 4 | 1.0513E+03 | monoisotopic | 4201 | 341 | up |
| | 5 | 8.4127E+02 | monoisotopic | 4201 | 341 | |
| 7* | 1 | 4.9723E+02 | monoisotopic | 496 | 279 | down |
| 8 | 3 | 1.1113E+03 | monoisotopic | 3331 | 452 | up |
| | 4 | 8.3369E+02 | monoisotopic | 3331 | 452 | |
| | 5 | 6.6715E+02 | monoisotopic | 3331 | 452 | |
| 9 | 3 | 7.2164E+02 | monoisotopic | 2162 | 495 | up |
| | 4 | 5.4148E+02 | monoisotopic | 2162 | 495 | |
| 10 | 6 | 1.0291E+03 | average | 6169 | 452 | up |
| | 7 | 8.8222E+02 | average | 6169 | 452 | |
| | 8 | 7.7207E+02 | average | 6169 | 452 | |
| 11 | 4 | 8.2773E+02 | monoisotopic | 3307 | 331 | up |
| 12 | 7 | 1.3279E+03 | average | 9288 | 643 | up |
| | 8 | 1.1620E+03 | average | 9288 | 643 | |
| | 9 | 1.0330E+03 | average | 9288 | 643 | |
| | 10 | 9.2982E+02 | average | 9288 | 643 | |
| 13 | 7 | 1.1050E+03 | average | 7728 | 400 | up |
| | 8 | 9.6701E+02 | average | 7728 | 400 | |
| | 9 | 8.5967E+02 | average | 7728 | 400 | |
| 14 | 7 | 1.3279E+03 | average | 9289 | 633 | up |
| | 8 | 1.1621E+03 | average | 9289 | 633 | |
| | 9 | 1.0331E+03 | average | 9289 | 633 | |
| | 10 | 9.2986E+02 | average | 9289 | 633 | |
| 15 | 4 | 8.0696E+02 | monoisotopic | 3224 | 564 | up |
| | 5 | 6.4576E+02 | monoisotopic | 3224 | 564 | |
| 16 | 1 | 7.6536E+02 | monoisotoplc | 764 | 235 | down |
| | 2 | 3.8318E+02 | monoisotopic | 764 | 235 | |
| 17* | 1 | 6.1935E+02 | monoisotopic | 618 | 265 | up |
| 18 | 6 | 9.5430E+02 | average | 5720 | 483 | up |
| | 7 | 8.1812E+02 | average | 5720 | 483 | |
| | 8 | 7.1598E+02 | average | 5720 | 483 | |
| | 9 | 6.3653E+02 | average | 5720 | 483 | |

TABLE 8B

| Biomarker | | | | | Separation | |
|---|---|---|---|---|---|---|
| (*molecular weight for the | | | | | Time (sec) | up or down |
| indicated monoisotopic | | Observed m/z | monoisotopic* or | Molecular Weight | (+/−64 sec for | regulated in |
| entities is as shown or +1 dalton) | Charge | (thomson) | average for m/z | (Daltons) | 95% CI) | cancer cells |
| 19 | 2 | 6.9929E+02 | monoisotopic | 1397 | 246 | up |
| 20 | 12 | 9.5422E+02 | average | 11439 | 482 | up |
| | 13 | 8.8089E+02 | average | 11439 | 482 | |
| | 14 | 8.1804E+02 | average | 11439 | 482 | |
| | 15 | 7.6357E+02 | average | 11439 | 482 | |
| | 16 | 7.1591E+02 | average | 11439 | 482 | |
| | 17 | 6.7386E+02 | average | 11439 | 482 | |
| | 18 | 6.3648E+02 | average | 11439 | 482 | |
| 21 | 13 | 1.0812E+03 | average | 14043 | 451 | up |
| | 14 | 1.0040E+03 | average | 14043 | 451 | |
| | 15 | 9.3718E+02 | average | 14043 | 451 | |
| | 16 | 8.7867E+02 | average | 14043 | 451 | |
| | 17 | 8.2704E+02 | average | 14043 | 451 | |
| | 18 | 7.8115E+02 | average | 14043 | 451 | |
| | 19 | 7.4009E+02 | average | 14043 | 451 | |
| 22 | 3 | 5.4295E+02 | monoisotopic | 1626 | 470 | up |
| | 4 | 4.0747E+02 | monoisotopic | 1626 | 470 | |
| 23* | 1 | 3.3413E+02 | monoisotopic | 333 | 296 | up |
| 24 | 13 | 1.0569E+03 | average | 13727 | 455 | up |
| | 14 | 9.8152E+02 | average | 13727 | 455 | |
| | 15 | 9.1615E+02 | average | 13727 | 455 | |
| | 16 | 8.5896E+02 | average | 13727 | 455 | |
| | 17 | 8.0849E+02 | average | 13727 | 455 | |
| | 18 | 7.6363E+02 | average | 13727 | 455 | |
| | 19 | 7.2349E+02 | average | 13727 | 455 | |
| 25 | 14 | 9.9214E+02 | average | 13876 | 494 | up |
| | 15 | 9.2607E+02 | average | 13876 | 494 | |
| | 16 | 8.6825E+02 | average | 13876 | 494 | |
| | 17 | 8.1723E+02 | average | 13876 | 494 | |
| | 18 | 7.7189E+02 | average | 13876 | 494 | |
| 26* | 1 | 2.2911E+02 | monoisotopic | 228 | 193 | down |
| 27* | 1 | 3.2712E+02 | monoisotopic | 326 | 194 | up |
| 28 | 2 | 4.8368E+02 | monoisotopic | 965 | 199 | up |
| 29* | 1 | 2.5715E+02 | monoisotopic | 256 | 199 | down |
| 30 | 1 | 6.2533E+02 | monoisotopic | 624 | 306 | up |
| | 2 | 3.1316E+02 | monoisotopic | 624 | 306 | |
| | 3 | 2.0911E+02 | monoisotopic | 624 | 306 | |
| 31 | 2 | 4.4813E+02 | monoisotopic | 894 | 235 | down |

TABLE 8C

| Biomarker | | | | | Separation | |
|---|---|---|---|---|---|---|
| (*molecular weight for the | | | | | Time (sec) | up or down |
| indicated monoisotopic | | Observed m/z | monoisotopic* or | Molecular Weight | (+/−64 sec for | regulated in |
| entities is as shown or +1 dalton) | Charge | (thomson) | average for m/z | (Daltons) | 95% CI) | cancer cells |
| 32 | 1 | 8.5739E+02 | monoisotopic | 856 | 235 | down |
| | 2 | 4.2920E+02 | monoisotopic | 856 | 235 | |
| 33 | 7 | 1.7797E+03 | average | 12451 | 373 | up |
| | 8 | 1.5574E+03 | average | 12451 | 373 | |
| | 9 | 1.3845E+03 | average | 12451 | 373 | |
| 34 | 3 | 6.1932E+02 | monoisotopic | 1855 | 328 | up |
| 35 | 10 | 1.1739E+03 | average | 11729 | 601 | up |
| | 11 | 1.0673E+03 | average | 11729 | 601 | |
| | 12 | 9.7840E+02 | average | 11729 | 601 | |
| | 13 | 9.0322E+02 | average | 11729 | 601 | |
| | 14 | 8.3878E+02 | average | 11729 | 601 | |
| 36 | 13 | 1.0700E+03 | average | 13897 | 451 | up |
| | 14 | 9.9366E+02 | average | 13897 | 451 | |
| | 15 | 9.2748E+02 | average | 13897 | 451 | |
| | 16 | 8.6957E+02 | average | 13897 | 451 | |
| | 17 | 8.1848E+02 | average | 13897 | 451 | |
| | 18 | 7.7307E+02 | average | 13897 | 451 | |
| | 19 | 7.3243E+02 | average | 13897 | 451 | |
| | 20 | 6.9586E+02 | average | 13897 | 451 | |
| 37 | 11 | 1.2593E+03 | average | 13841 | 443 | up |
| | 12 | 1.1544E+03 | average | 13841 | 443 | |
| | 13 | 1.0657E+03 | average | 13841 | 443 | |
| | 14 | 9.8967E+02 | average | 13841 | 443 | |
| | 15 | 9.2376E+02 | average | 13841 | 443 | |
| | 16 | 8.6609E+02 | average | 13841 | 443 | |
| | 17 | 8.1520E+02 | average | 13841 | 443 | |
| | 18 | 7.6997E+02 | average | 13841 | 443 | |
| | 19 | 7.2949E+02 | average | 13841 | 443 | |

TABLE 8D

| Biomarker | | | | | Separation | |
|---|---|---|---|---|---|---|
| (*molecular weight for the | | | | | Time (sec) | up or down |
| indicated monoisotopic | | Obeserved m/z | monoisotopic* or | Molecular Weight | (+/−64 sec for | regulated in |
| entities is as shown or +1 dalton) | Charge | (thomson) | average for m/z | (Daltons) | 95% CI) | cancer cells |
| 38 | 11 | 1.2717E+03 | average | 13978 | 452 | up |
| | 12 | 1.1659E+03 | average | 13978 | 452 | |
| | 13 | 1.0762E+03 | average | 13978 | 452 | |
| | 14 | 9.9944E+02 | average | 13978 | 452 | |
| | 15 | 9.3288E+02 | average | 13978 | 452 | |
| | 16 | 8.7464E+02 | average | 13978 | 452 | |
| | 17 | 8.2325E+02 | average | 13978 | 452 | |
| | 18 | 7.7757E+02 | average | 13978 | 452 | |
| 39 | 6 | 1.1060E+03 | average | 6630 | 585 | up |
| | 7 | 9.4818E+02 | average | 6630 | 585 | |
| | 8 | 8.2978E+02 | average | 6630 | 585 | |
| | 9 | 7.3769E+02 | average | 6630 | 585 | |
| | 10 | 6.6402E+02 | average | 6630 | 585 | |
| | 11 | 6.0375E+02 | average | 6630 | 585 | |
| 40* | 1 | 6.8650E+02 | monoisotopic | 686 | 195 | up |
| 41* | 1 | 3.1314E+02 | monoisotopic | 312 | 305 | up |
| 42 | 2 | 7.3335E+02 | monoisotopic | 1465 | 266 | down |
| | 3 | 4.8924E+02 | monoisotopic | 1465 | 266 | |
| | 4 | 3.6718E+02 | monoisotopic | 1465 | 266 | |
| 43 | 2 | 4.9167E+02 | monoisotopic | 981 | 198 | up |
| 44 | 1 | 9.4442E+02 | monoisotopic | 943 | 198 | up |
| | 2 | 4.7271E+02 | monoisotopic | 943 | 198 | |
| 45* | 1 | 2.7310E+02 | monoisotopic | 272 | 192 | down |
| 46* | 1 | 229.1146625 | monoisotopic | 228 | 337 | down |
| 47* | 1 | 342.145859 | monoisotopic | 341 | 440 | up |

The above examples are in no way intended to limit the scope of the invention. Further, it can be appreciated to one of ordinary skill in the art that many changes and modifications can be made thereto without departing from the spirit or scope of the appended claims, and such changes and modifications are contemplated within the scope of the instant invention.

All publications, patents, and patent applications mentioned in this specification are herein incorporated by reference to the same extent as if each individual publication, patent, or patent application was specifically and individually indicated to be incorporated by reference.

What is claimed is:

1. A method of analyzing a prostate cancer state of a patient comprising:
identifying a first subset of prostate cancer markers in a biological sample, wherein said markers in said first subset comprise at least one marker from a first set of prostate cancer markers, said markers in said first set being that marker that can provide mass spectral signals selected from following approximate m/z values:

| Biomarker | |
|---|---|
| (*molecular weight | |
| for the indicated | |
| entity is as shown or | Observed m/z |
| +1 dalton) | (thomson) |
| 1* | 2.9511E+02 |
| 2 | 1.5433E+03 |
| | 1.3890E+03 |
| | 1.2629E+03 |
| | 1.1577E+03 |
| | 1.0687E+03 |
| | 9.9246E+02 |
| | 9.2636E+02 |
| | 8.6852E+02 |
| | 8.1749E+02 |
| | 7.7213E+02 |
| | 7.3155E+02 |
| | 6.9502E+02 |
| | 6.6197E+02 |

and
providing information regarding a prostate cancer state, wherein said information is based on a review of said first subset of prostate cancer markers.

2. The method of claim 1 wherein said markers in said first set are further characterized by following molecular weights and charge states:

| Biomarker | | | |
|---|---|---|---|
| (*molecular weight | | | |
| for the indicated | | | |
| monoisotopic | | | |
| entities is as | | | Molecular |
| shown or +1 | | Observed m/z | Weight |
| dalton) | Charge | (thomson) | (Daltons) |
| 1* | 1 | 2.9511E+02 | 294 |
| 2 | 9 | 1.5433E+03 | 13880 |
| | 10 | 1.3890E+03 | 13880 |
| | 11 | 1.2629E+03 | 13880 |
| | 12 | 1.1577E+03 | 13880 |
| | 13 | 1.0687E+03 | 13880 |
| | 14 | 9.9246E+02 | 13880 |
| | 15 | 9.2636E+02 | 13880 |
| | 16 | 8.6852E+02 | 13880 |
| | 17 | 8.1749E+02 | 13880 |
| | 18 | 7.7213E+02 | 13880 |
| | 19 | 7.3155E+02 | 13880 |
| | 20 | 6.9502E+02 | 13880 |
| | 21 | 6.6197E+02 | 13880 |

3. The method of claim 2 further comprising:
identifying a second subset of prostate cancer markers in a biological sample from a second set of prostate cancer markers, said second set comprising at least one marker selected from prostate specific antigen, human glandular kallikrein 2, prostatic acid phosphatase, prostate-specific membrane antigen, androgen receptor, insulin-like growth factor, and insulin-like growth factor binding protein.

4. The method of claim 1 wherein said markers are identified using a mass spectrometry system.

5. The method of claim 4 wherein said mass spectrometry system is a time-of-flight mass spectrometry system.

6. The method of claim 4 wherein said biological sample is separated using microchannel electrophoresis or capillary electrophoresis on a chip format.

7. The method of claim 4 wherein said biological sample is prepared and/or separated on a microfluidics device.

8. The method of claim 4 wherein said biological sample is delivered to said mass spectrometry system by electrospray ionization.

9. The method of claim 4 wherein said biological sample is delivered to said mass spectrometry system by matrix assisted laser desorption ionization.

10. The method of claim 1 wherein said markers are identified using at least one technique selected from an antibody-based technique, a multiplexed antibody array, a multiplexed antibody bead, a protein affinity chip, an aptamer, and a microsequencing technique.

11. A diagnostic product for prostate cancer comprising at least one component adapted and configured for performing the method as recited in claim 1.

12. A method of analyzing a prostate cancer state of a patient comprising:
reviewing a pattern of prostate cancer markers from a patient, said pattern comprising at least one marker from a set of markers that can provide mass spectral signals selected from following approximate m/z values:

| Biomarker | |
|---|---|
| (*molecular weight | |
| for the indicated | |
| entity is as shown or | Observed m/z |
| +1 dalton) | (thomson) |
| 1* | 2.9511E+02 |
| 2 | 1.5433E+03 |
| | 1.3890E+03 |
| | 1.2629E+03 |
| | 1.1577E+03 |
| | 1.0687E+03 |
| | 9.9246E+02 |
| | 9.2636E+02 |
| | 8.6852E+02 |
| | 8.1749E+02 |
| | 7.7213E+02 |
| | 7.3155E+02 |
| | 6.9502E+02 |
| | 6.6197E+02 |

and providing an information regarding a prostate cancer state to said patient, a health care provider or a health care manager, said information being based on said review of pattern.

13. The method of claim 12 wherein said markers in said set are further characterized by following approximate molecular weights and charge states:

| Biomarker | | | |
|---|---|---|---|
| (*molecular | | | |
| weight for the | | | |
| indicated | | | |
| monoisotopic | | | |
| entities is as | | | Molecular |
| shown or +1 | | Observed m/z | Weight |
| dalton) | Charge | (thomson) | (Daltons) |
| 1* | 1 | 2.9511E+02 | 294 |
| 2 | 9 | 1.5433E+03 | 13880 |
| | 10 | 1.3890E+03 | 13880 |
| | 11 | 1.2629E+03 | 13880 |
| | 12 | 1.1577E+03 | 13880 |
| | 13 | 1.0687E+03 | 13880 |
| | 14 | 9.9246E+02 | 13880 |
| | 15 | 9.2636E+02 | 13880 |
| | 16 | 8.6852E+02 | 13880 |
| | 17 | 8.1749E+02 | 13880 |
| | 18 | 7.7213E+02 | 13880 |
| | 19 | 7.3155E+02 | 13880 |
| | 20 | 6.9502E+02 | 13880 |
| | 21 | 6.6197E+02 | 13880 |

14. An computer-readable medium comprising:
a medium suitable for transmission of a result of an analysis of a biological sample; said medium comprising an information regarding a prostate cancer state of a subject, wherein said information is derived using the method of claim 1 or 12.

15. A method of analyzing a prostate cancer state of a patient comprising:
identifying a subset of prostate cancer markers in a biological sample, wherein said markers in said subset comprise at least one marker from a set of prostate cancer markers, said markers in said set being characterized by following molecular weights:

| Biomarker | |
|---|---|
| (*molecular | |
| weight for the | |
| indicated entities | |
| is as shown | Molecular Weight |
| or +1 dalton) | (Daltons) |
| 1* | 294 |
| 2 | 13880 |
| 3 | 1050 |
| 4 | 519 |
| 5 | 9061 |
| 6 | 4201 |
| 7* | 496 |
| 8 | 3331 |
| 9 | 2162 |
| 10 | 6169 |
| 11 | 3307 |
| 12 | 9288 |
| 13 | 7728 |
| 14 | 9289 |
| 15 | 3224 |
| 16 | 764 |
| 17* | 618 |
| 18 | 5720 |
| 19 | 1397 |
| 20 | 11439 |
| 21 | 14043 |
| 22 | 1626 |
| 23* | 333 |
| 24 | 13727 |
| 25 | 13876 |
| 26* | 228 |
| 27* | 326 |
| 28 | 965 |
| 29* | 256 |
| 30 | 624 |
| 31 | 894 |
| 32 | 856 |
| 33 | 12451 |
| 34 | 1855 |
| 35 | 11729 |
| 36 | 13897 |
| 37 | 13841 |
| 38 | 13978 |
| 39 | 6630 |
| 40* | 686 |
| 41* | 312 |
| 42 | 1465 |
| 43 | 981 |
| 44 | 943 |
| 45* | 272 |
| 46* | 228 |
| 47* | 341 |

and providing information regarding a prostate cancer state, said information being based on a review of said subset of prostate cancer markers.

16. The method of claim 15 wherein said markers in said first subset are further characterized by following charge states and/or m/z ratios:

| Biomarker | | | |
|---|---|---|---|
| (*molecular weight | | | |
| for the indicated | | | Molecular |
| monoisotopic entities is | | Observed m/z | Weight |
| as shown or +1 dalton) | Charge | (thomson) | (Daltons) |
| 3 | 2 | 5.2576E+02 | 1050 |
| 4 | 1 | 5.2035E+02 | 519 |
| | 2 | 2.6067E+02 | 519 |
| 5 | 8 | 1.1336E+03 | 9061 |
| | 9 | 1.0077E+03 | 9061 |
| | 10 | 9.0707E+02 | 9061 |
| 6 | 4 | 1.0513E+03 | 4201 |
| | 5 | 8.4127E+02 | 4201 |
| 7* | 1 | 4.9723E+02 | 496 |
| 8 | 3 | 1.1113E+03 | 3331 |
| | 4 | 8.3369E+02 | 3331 |
| | 5 | 6.6715E+02 | 3331 |
| 9 | 3 | 7.2164E+02 | 2162 |
| | 4 | 5.4148E+02 | 2162 |
| 10 | 6 | 1.0291E+03 | 6169 |
| | 7 | 8.8222E+02 | 6169 |
| | 8 | 7.7207E+02 | 6169 |
| 11 | 4 | 8.2773E+02 | 3307 |
| 12 | 7 | 1.3279E+03 | 9288 |
| | 8 | 1.1620E+03 | 9288 |
| | 9 | 1.0330E+03 | 9288 |
| | 10 | 9.2982E+02 | 9288 |
| 13 | 7 | 1.1050E+03 | 7728 |
| | 8 | 9.6701E+02 | 7728 |
| | 9 | 8.5967E+02 | 7728 |
| 14 | 7 | 1.3279E+03 | 9289 |
| | 8 | 1.1621E+03 | 9289 |
| | 9 | 1.0331E+03 | 9289 |
| | 10 | 9.2986E+02 | 9289 |
| 15 | 4 | 8.0696E+02 | 3224 |
| | 5 | 6.4576E+02 | 3224 |
| 16 | 1 | 7.6536E+02 | 764 |
| | 2 | 3.8318E+02 | 764 |
| 17* | 1 | 6.1935E+02 | 618 |
| 18 | 6 | 9.5430E+02 | 5720 |
| | 7 | 8.1812E+02 | 5720 |
| | 8 | 7.1598E+02 | 5720 |
| | 9 | 6.3653E+02 | 5720 |
| 19 | 2 | 6.9929E+02 | 1397 |
| 20 | 12 | 9.5422E+02 | 11439 |
| | 13 | 8.8089E+02 | 11439 |
| | 14 | 8.1804E+02 | 11439 |
| | 15 | 7.6357E+02 | 11439 |
| | 16 | 7.1591E+02 | 11439 |
| | 17 | 6.7386E+02 | 11439 |
| | 18 | 6.3648E+02 | 11439 |
| 21 | 13 | 1.0812E+03 | 14043 |
| | 14 | 1.0040E+03 | 14043 |
| | 15 | 9.3718E+02 | 14043 |
| | 16 | 8.7867E+02 | 14043 |
| | 17 | 8.2704E+02 | 14043 |
| | 18 | 7.8115E+02 | 14043 |
| | 19 | 7.4009E+02 | 14043 |
| 22 | 3 | 5.4295E+02 | 1626 |
| | 4 | 4.0747E+02 | 1626 |
| 23* | 1 | 3.3413E+02 | 333 |
| 24 | 13 | 1.0569E+03 | 13727 |
| | 14 | 9.8152E+02 | 13727 |
| | 15 | 9.1615E+02 | 13727 |
| | 16 | 8.5896E+02 | 13727 |
| | 17 | 8.0849E+02 | 13727 |
| | 18 | 7.6363E+02 | 13727 |
| | 19 | 7.2349E+02 | 13727 |
| 25 | 14 | 9.9214E+02 | 13876 |
| | 15 | 9.2607E+02 | 13876 |
| | 16 | 8.6825E+02 | 13876 |
| | 17 | 8.1723E+02 | 13876 |
| | 18 | 7.7189E+02 | 13876 |
| 26* | 1 | 2.2911E+02 | 228 |
| 27* | 1 | 3.2712E+02 | 326 |
| 28 | 2 | 4.8368E+02 | 965 |
| 29* | 1 | 2.5715E+02 | 256 |
| 30 | 1 | 6.2533E+02 | 624 |
| | 2 | 3.1316E+02 | 624 |
| | 3 | 2.0911E+02 | 624 |
| 31 | 2 | 4.4813E+02 | 894 |
| 32 | 1 | 8.5739E+02 | 856 |
| | 2 | 4.2920E+02 | 856 |
| 33 | 7 | 1.7797E+03 | 12451 |
| | 8 | 1.5574E+03 | 12451 |
| | 9 | 1.3845E+03 | 12451 |
| 34 | 3 | 6.1932E+02 | 1855 |
| 35 | 10 | 1.1739E+03 | 11729 |
| | 11 | 1.0673E+03 | 11729 |
| | 12 | 9.7840E+02 | 11729 |
| | 13 | 9.0322E+02 | 11729 |
| | 14 | 8.3878E+02 | 11729 |
| 36 | 13 | 1.0700E+03 | 13897 |
| | 14 | 9.9366E+02 | 13897 |
| | 15 | 9.2748E+02 | 13897 |
| | 16 | 8.6957E+02 | 13897 |
| | 17 | 8.1848E+02 | 13897 |
| | 18 | 7.7307E+02 | 13897 |
| | 19 | 7.3243E+02 | 13897 |
| | 20 | 6.9586E+02 | 13897 |
| 37 | 11 | 1.2593E+03 | 13841 |
| | 12 | 1.1544E+03 | 13841 |
| | 13 | 1.0657E+03 | 13841 |
| | 14 | 9.8967E+02 | 13841 |
| | 15 | 9.2376E+02 | 13841 |
| | 16 | 8.6609E+02 | 13841 |
| | 17 | 8.1520E+02 | 13841 |
| | 18 | 7.6997E+02 | 13841 |
| | 19 | 7.2949E+02 | 13841 |
| 38 | 11 | 1.2717E+03 | 13978 |
| | 12 | 1.1659E+03 | 13978 |
| | 13 | 1.0762E+03 | 13978 |
| | 14 | 9.9944E+02 | 13978 |
| | 15 | 9.3288E+02 | 13978 |
| | 16 | 8.7464E+02 | 13978 |
| | 17 | 8.2325E+02 | 13978 |
| | 18 | 7.7757E+02 | 13978 |
| 39 | 6 | 1.1060E+02 | 6630 |
| | 7 | 9.4818E+02 | 6630 |
| | 8 | 8.2978E+02 | 6630 |
| | 9 | 7.3769E+02 | 6630 |
| | 10 | 6.6402E+02 | 6630 |
| | 11 | 6.0375E+02 | 6630 |
| 40* | 1 | 6.8650E+02 | 686 |
| 41* | 1 | 3.1314E+02 | 312 |
| 42 | 2 | 7.3335E+02 | 1465 |
| | 3 | 4.8924E+02 | 1465 |
| | 4 | 3.6718E+02 | 1465 |
| 43 | 2 | 4.9167E+02 | 981 |
| 44 | 1 | 9.4442E+02 | 943 |
| | 2 | 4.7271E+02 | 943 |
| 45* | 1 | 2.7310E+02 | 272 |
| 46* | 1 | 2.2911E+02 | 228 |
| 47* | 1 | 3.4215E+02 | 341 |

17. A method of analyzing prostate cancer states comprising identifying the presence of transthyretin, fragments of transthyretin, and/or post-translationally modified forms of transthyretin in a biological sample and making a decision regarding a prostate cancer state, wherein said biological sample is obtained from a subject with serum PSA levels of less than about 6 ng/ml.

18. A method of analyzing a prostate cancer state of a patient comprising:
identifying a subset of prostate cancer markers in a biological sample, wherein said markers said subset comprise at least one marker from a set of prostate cancer markers, said markers said set being identified with mass spectroscopy and said markers in said set being characterized by following molecular weights:

| Biomarker | |
|---|---|
| (*molecular | |
| weight for the | |
| indicated entities | Molecular Weight |
| is as shown or +1 dalton) | (Daltons) |
| 1* | 294 |
| 2 | 13880 |
| 3 | 1050 |
| 4 | 519 |
| 5 | 9061 |
| 6 | 4201 |
| 7* | 496 |
| 8 | 3331 |
| 9 | 2162 |
| 10 | 6169 |
| 11 | 3307 |
| 12 | 9288 |
| 13 | 7728 |
| 14 | 9289 |
| 15 | 3224 |
| 16 | 764 |
| 17* | 618 |
| 18 | 5720 |
| 19 | 1397 |
| 20 | 11439 |
| 21 | 14043 |
| 22 | 1626 |
| 23* | 333 |
| 24 | 13727 |
| 25 | 13876 |
| 26* | 228 |
| 27* | 326 |
| 28 | 965 |
| 29* | 256 |
| 30 | 624 |
| 31 | 894 |
| 32 | 856 |
| 33 | 12451 |
| 34 | 1855 |
| 35 | 11729 |
| 36 | 13897 |
| 37 | 13841 |
| 38 | 13978 |
| 39 | 6630 |
| 40* | 686 |
| 41* | 312 |
| 42 | 1465 |
| 43 | 981 |
| 44 | 943 |
| 45* | 272 |
| 46* | 228 |
| 47* | 341 |

and providing information regarding a prostate cancer state, said information being based on a review of said subset of prostate cancer markers.
